(19) United States
(12) Patent Application Publication
(10) Pub. No.: US 2021/0156844 A1
(43) Pub. Date: May 27, 2021

(54) TMEM16A Modulation for Diagnostic or Therapeutic Use in Pulmonary Hypertension (PH)

(71) Applicant: Bayer Aktiengesellschaft, Leverkusen (DE)

(72) Inventors: Andrea Olschewski, Graz (CH); Bence Nagy, Graz (CH); Chandran Nagaraj, Graz (CH); Rita Papp, Graz (CH)

(21) Appl. No.: 16/610,871

(22) PCT Filed: Apr. 24, 2018
(86) PCT No.: PCT/EP2018/060415

§ 371 Date: Nov. 4, 2019

(30) Foreign Application Priority Data
May 2, 2017 (EP) 17169063.9

(51) Int. Cl. G01N 33/50; A61K 31/343; A61K 31/713; G01N 33/68

(52) U.S. Cl. CPC G01N 33/5008; A61K 31/343; A61K 31/713; G01N 2800/54; G01N 2500/04; G01N 2800/12; G01N 2800/52; G01N 33/6872

(57) ABSTRACT

Provided is a method of assessing the occurrence or the risk of occurrence of pulmonary hypertension (PH), in which an increased level of expression of the channel TMEM16A indicates a risk of occurrence of PH. Provided is also a method of treating PH, in which a compound effective in reducing activity of TMEM16A is being administered. Provided are also screening methods for a compound suitable for modulating activity ex vivo and in vitro and for PH treatment in vivo.

### FIELD OF THE DISCLOSURE

Provided are methods of diagnosing and treating pulmonary arterial hypertension (PAH) in a subject. Disclosed are also methods of identifying a compound suitable for modulating the activity of TMEM16A.

### BACKGROUND

The following discussion of the background is merely provided to aid the reader in understanding the invention and is not admitted to describe or constitute prior art to the present invention.

Pulmonary hypertension (PH) is a life-threatening chronic disorder of the pulmonary circulation. PH is a progressive disease leading to decreased exercise capacity, right heart failure, and ultimately death. It is a haemodynamic abnormality of diverse aetiology and pathogenesis that challenges physicians with both its diagnosis and treatment. PH is clinically defined as a resting mean pulmonary arterial pressure ≥25 mmHg measured by right heart catheterization. The prognosis is poor, without specific treatment 1-, 3- and 5-year survivals are 68, 48 and 34%, respectively.

The disease is characterized by the constriction of precapillary pulmonary arteries, associated with irreversible remodeling. The resulting increase in the pulmonary arterial pressure leads to right ventricular hypertrophy and eventually death from right heart failure. Excess proliferation of pulmonary arterial endothelial and smooth muscle cells (SMC) is one of the final, common pathological outcome of distinct pathways involved in the development of pulmonary arterial hypertension (PAH).

Pulmonary arterial hypertension (PAH) is a vascular disease that defines a category of PH, namely Group I PH according to the WHO classification, and involves an increase of blood pressure in small pulmonary arteries. Furthermore it involves the formation of obstructed, constricted small pulmonary arteries, associated with irreversible remodeling.

As a result of the constriction of precapillary pulmonary arteries, precapillary pulmonary hypertension occurs, with elevated pulmonary vascular resistance, i.e. a mean pulmonary artery pressure of ≥25 mm Hg. In addition, PAH is defined by a normal pulmonary arterial wedge pressure ≤15 mm Hg and pulmonary vascular resistance of >240 dyn×s×cm⁻⁵. Initially PAH was thought to be a disease that mostly affected young women. However, the mean age of patients diagnosed with PAH in Germany has steadily increased; presently the mean age is 65 years (Hoeper, M M, et al. Dtsch Arztebl Int (2017) 114: 73-84).

The increase in the pulmonary arterial pressure leads to right ventricular hypertrophy and eventually death from right heart failure. Excess proliferation of pulmonary arterial endothelial and smooth muscle cells (SMC) is one of the final, common pathological outcomes of distinct pathways involved in the development of PAH.

Pulmonary hypertension occurs in different forms and is expected to be based on multiple causes. ‘Current Medical Diagnosis & Treatment’ (55 edition, 2016, McGraw-Hill Education, 425-429) classifies the disease into 5 groups, with PAH related to an underlying pulmonary vasculopathy falling under Group 1. It includes the former “primary” pulmonary hypertension under the term “idiopatic pulmonary arterial hypertension” (IPAH). The median survival time for patients with PAH in the US is presently 7 years, and current median survival for IPAH patients in the US is 2.8 years.

PH can be a cost-intensive disease and can impose a substantial burden on healthcare resources. The treatment of pulmonary hypertension is predominantly symptomatic and depends on the type and severity of the disease and the patient's requirements. Recommendations with regard to targeted therapy of PAH in the guidelines of the European Society of Cardiology (ESC) and the European Respiratory Society (ERS) are based on an individual risk assessment. Current methods of treating PH include diuretics, and where required oxygen therapy. Treatment of IPAH further includes anticoagulation, as well as acute vasodilator testing, unless contraindicated. There thus remains a need for an effective treatment of pulmonary hypertension, including pulmonary arterial hypertension. The mechanisms of PAH, including IPAH, remain unclear.

### SUMMARY

The present disclosure provides methods that allow diagnosing and treating PH. Provided are also screening methods for an agent suitable for modulating TMEM16A and for an agent suitable for PH treatment. A method and use in therapy of PH, as disclosed herein, allows the treatment of both the increased pulmonary arterial pressure and the vascular remodeling that occur in PH. Accordingly, reverse pulmonary vascular remodelling can be achieved using a respective method or use. Without being bound by theory, treatment according to a method and use as disclosed herein can be taken to rely on inhibition of the calcium-activated chloride channel including TMEM16A.

According to a first aspect, there is provided a method of assessing the occurrence or the risk of occurrence of pulmonary hypertension (PH) in a subject. Accordingly, the method is in some cases a method of assessing the occurrence of PH, i.e. assessing whether a subject is suffering from PH. In some cases the method is a method of assessing the risk of occurrence of pulmonary hypertension (PH) in a subject. In the latter cases, the method may also be taken to be a method of predicting the likelihood that PH will occur in the subject. In any case, the method includes detecting the expression level of the channel TMEM16A in a sample from the subject. An increased level of TMEM6A expression, relative to a threshold value, indicates an elevated risk of occurrence of PH. An unchanged or a reduced level of TMEM16A expression indicates no elevated risk of occurrence of PH. An increased level of TMEM16A expression, relative to a threshold value, also indicates the likelihood of occurrence of PH in the subject. An unchanged or a reduced level of TMEM16A expression indicates no likelihood of occurrence of PH in the subject.

Generally the method includes determining if the level of TMEM16A in the sample from the subject is different from the threshold value, e.g. from the level of a reference TMEM6A expression.

Typically TMEM16A is a protein encoded by an ANO1 gene of the subject.

In some embodiments the method essentially consists of detecting the expression level of TMEM16A in a sample from the subject and comparing the expression level to a threshold value.

In some embodiments the PH is pulmonary arterial hypertension (PAH). In some embodiments the PAH is Group 1 PAH. The Group 1 PAH may for example be or include idiopathic or primary pulmonary hypertension. The Group 1 PAH may in some embodiments also be or involve familial hypertension. In some embodiments the Group 1 PAH may include or may be pulmonary hypertension secondary to chronic hypoxia. In some embodiments the Group 1 PAH may include or may be pulmonary hypertension secondary to, but not limited to, connective tissue disease, congenital heart defects (shunts), pulmonary fibrosis, portal hypertension, HIV infection, sickle cell disease, a drug and/or a toxin (e.g., anorexigens, cocaine chronic pulmonary obstructive disease, sleep apnea, and schistosomiasis. In some embodiments the Group 1 PAH may include or may be pulmonary hypertension associated with significant venous or capillary involvement (pulmonary veno-occlusive disease, pulmonary capillary hemangiomatosis). In some embodiments the Group 1 PAH may include or may be pulmonary hypertension associated with secondary pulmonary hypertension that is out of proportion to the degree of left ventricular dysfunction. In some embodiments the Group 1 PAH may include or may be persistent pulmonary hypertension in a newborn baby.

In some embodiments the subject is human. In some embodiment the subject is a horse or a pig. In some embodiments the subject is a dog. The sample from the subject is in some embodiments a pulmonary arterial smooth muscle sample.

The threshold value, compared to which the level of TMEM16A expression is increased, is in some embodiments an average value based on an assessment, for example a previous assessment, of healthy subjects of the same species, e.g. humans. In some embodiments the threshold value is a value determined from the same subject at an earlier point in time. The threshold value may also be based on a reference sample. The reference sample may be from one or more healthy subjects of the same species. The reference sample may also be from the same subject taken at an earlier point in time.

According to a second aspect, there is provided a method of identifying an agent capable of modulating activity of TMEM16A. The method includes contacting in vitro or ex vivo TMEM16A with an agent suspected to modulate activity of TMEM16A. The method further includes detecting the activity of TMEM16A. In some embodiments the method essentially consists of contacting in vitro or ex vivo TMEM16A with an agent suspected to modulate activity of TMEM16A, and detecting the activity of TMEM16A. In some embodiments the method consists of contacting in vitro or ex vivo TMEM16A with a respective agent, and detecting the activity of TMEM16A.

Typically an agent capable of modulating activity of TMEM16A is an agent capable of reducing and/or inhibiting activity of TMEM16A.

Typically TMEM6A is a protein encoded by an ANO1 gene of a mammal. In some embodiments TMEM16A is a protein encoded by the human ANO1 gene.

In some embodiments of the method according to the second aspect, TMEM16A has been expressed by a host cell. In some embodiments TMEM16A is comprised in a host cell. The host cell may for example be a pulmonary arterial smooth muscle cell.

The agent capable of modulating activity of TMEM16A is generally a compound. The agent capable of modulating activity of TMEM16A is in some embodiments a low molecular weight compound or a pharmaceutically acceptable salt thereof. In some embodiments the agent capable of modulating activity of TMEM16A is an antibody binding to TMEM16A. An agent capable of modulating activity of TMEM16A is in some embodiments an agent capable of reducing, including inhibiting, activity of TMEM16A. In some embodiments, the method according to the second aspect is a method of identifying an agent capable of reducing, including inhibiting, activity of TMEM16A.

In some embodiments of the method according to the second aspect, detecting the activity of TMEM6A includes comparing the activity of TMEM6A to a control measurement. In some embodiments detecting the activity of TMEM16A includes comparing the activity of TMEM16A to a threshold value. In some embodiments detecting the activity of TMEM16A includes comparing the activity of TMEM16A to both a control measurement and to a threshold value.

According to a related third aspect, there is provided the use of the protein TMEM16A for identifying an agent suitable for modulating activity of TMEM16A. The use includes contacting TMEM16A in vitro or ex vivo with an agent suspected to modulate activity of TMEM16A. The method further includes detecting the activity of TMEM16A. In some embodiments the use essentially consists of contacting in vitro or ex vivo TMEM16A with an agent suspected to modulate activity of TMEM16A, and detecting the activity of TMEM16A. In some embodiments the use consists of contacting in vitro or ex vivo TMEM16A with a respective agent, and detecting the activity of TMEM16A.

As noted above, an agent capable of modulating activity of TMEM16A is typically an agent capable of reducing and/or inhibiting activity of TMEM16A.

In some embodiments, the use according to the third aspect is a use in identifying an agent capable of reducing, including inhibiting, activity of TMEM16A. The agent capable of modulating activity of TMEM16A is generally a compound or a pharmaceutically acceptable salt thereof. In some embodiments the agent capable of modulating activity of TMEM16A is a low molecular weight compound or a pharmaceutically acceptable salt thereof. In some embodiments the agent capable of modulating activity of TMEM16A is an antibody binding to TMEM16A.

According to a fourth aspect, there is provided the use of a nonhuman animal to screen an agent for activity in treating PH. The nonhuman animal is an animal that has been exposed to hypoxic conditions for at least 10 days, including for at least 12 days.

In some embodiments the use according to the fourth aspect includes determining the right ventricular systolic pressure (RVSP). A decrease in RVSP indicates that the agent is effective in treating PH. An unchanged RVSP or an increase in RVSP indicate that the agent is not effective in treating PH.

Screening an agent for activity in treating PH may involve screening an agent for preventing PH. Screening an agent for activity in treating PH may also involve screening an agent for inhibiting PH or for inhibiting the progress of PH. Screening an agent for activity in treating PH may also involve screening an agent for reversing PH.

In some embodiments of the use according to the fourth aspect, the nonhuman animal is an animal that has been exposed to hypoxic conditions for at least 14 days.

The nonhuman animal used according to the fourth aspect may in some embodiments be a mouse or a rat. In some embodiments the nonhuman animal may be a guinea pig or a hamster. In some embodiments the nonhuman animal is an ape or a monkey. The agent to be screened is in some embodiments a low molecular weight compound or a pharmaceutically acceptable salt thereof. In some embodiments the agent to be screened is an antibody binding to TMEM16A.

Screening an agent for activity in treating PH is generally screening an agent for activity in treating PH in a subject. In some embodiments the subject is human. In some embodiment the subject is a horse or a pig. In some embodiments the subject is a dog.

In some embodiments the PH that is to be treated, including prevented, is PAH. In some embodiments the PAH is Group 1 PAH. The Group 1 PAH may for example be or include idiopathic or primary pulmonary hypertension. The Group 1 PAH may in some embodiments also be or involve familial hypertension. In some embodiments the Group 1 PAH may include or may be pulmonary hypertension secondary to chronic hypoxia. In some embodiments the Group 1 PAH may include or may be pulmonary hypertension secondary to, but not limited to, connective tissue disease, congenital heart defects (shunts), pulmonary fibrosis, portal hypertension, HIV infection, sickle cell disease, a drug and/or a toxin (e.g., anorexigens, cocaine chronic pulmonary obstructive disease, sleep apnea, and schistosomiasis. In some embodiments the Group 1 PAH may include or may be pulmonary hypertension associated with significant venous or capillary involvement (pulmonary veno-occlusive disease, pulmonary capillary hemangiomatosis). In some embodiments the Group 1 PAH may include or may be pulmonary hypertension associated with secondary pulmonary hypertension that is out of proportion to the degree of left ventricular dysfunction. In some embodiments the Group 1 PAH may include or may be persistent pulmonary hypertension in a newborn baby.

According to a related fifth aspect, there is provided an in vivo method for identifying an agent that is effective in treating PH. The method includes administering the agent to a nonhuman animal. The nonhuman animal is an animal that has been exposed to hypoxic conditions for at least 10 days, including for at least 12 days. In some embodiments of the use according to the third aspect, the nonhuman animal is an animal that has been exposed to hypoxic conditions for at least 14 days.

The method according to the fifth aspect may involve identifying an agent that is effective in preventing PH. The method may also involve identifying an agent that is effective in inhibiting PH or in inhibiting the progress of PH. The method may also involve identifying an agent that is effective in reversing PH.

In some embodiments, the agent used in the method according to the fifth aspect is an agent that is suspected to be effective in reducing, including inhibiting, activity of TMEM16A. In some embodiments, the agent used in the method according to the fifth aspect is an agent that is known to be effective in reducing, including inhibiting, activity of TMEM16A.

The agent effective in treating PH, including preventing PH, is generally a compound or a pharmaceutically acceptable salt thereof. In some embodiments the agent effective in treating PH is a low molecular weight compound or a pharmaceutically acceptable salt thereof. In some embodiments the agent effective in treating PH is an antibody binding to TMEM16A.

The PH to be treated may be PAH. In some embodiments the PAH is Group 1 PAH. The Group 1 PAH may in some embodiments be idiopathic or primary pulmonary hypertension. As already indicated above, in some embodiments the Group 1 PAH may for example be or include a condition such as familial hypertension and/or another condition defined above. The Group 1 PAH may also be secondary to one or more of the conditions defined above.

In some embodiments, the method according to the fifth aspect is a method in identifying an agent capable of reducing, including inhibiting, activity of TMEM16A. As explained above, the agent capable of reducing, including inhibiting, activity of TMEM16A is generally a compound or a pharmaceutically acceptable salt thereof. In some embodiments the agent capable of reducing, including inhibiting, activity of TMEM16A is a low molecular weight compound or a pharmaceutically acceptable salt thereof. In some embodiments the agent capable of reducing, including inhibiting, activity of TMEM16A is an antibody binding to TMEM16A.

Identifying an agent that is effective in treating PH is generally identifying an agent that is effective in treating PH in a subject, including in treating PAH in a subject. In some embodiments the subject is human. In some embodiment the subject is a horse or a pig. In some embodiments the subject is a dog.

In some embodiments the method according to the fifth aspect includes determining the RVSP in the respective subject. A decrease in RVSP indicates that the agent is effective in treating PH. An unchanged RVSP or an increase in RVSP indicate that the agent is not effective in treating PH.

According to a sixth aspect, there is provided an agent suspected or known to reduce activity of TMEM16A for use in a screening method for treating PH. The respective screening method includes administration of the agent to a non-human animal. The non-human animal has been exposed to hypoxic conditions for at least 10 days, including for at least 12 days. The used screening method further includes determining the RVSP. As explained above, a decrease in RVSP indicates that the agent is effective in treating PH. An unchanged RVSP or an increase in RVSP indicate that the agent is not effective in treating PH.

The agent suspected or known to reduce activity of TMEM16A is generally a compound or a pharmaceutically acceptable salt thereof. In some embodiments the agent suspected or known to reduce activity of TMEM16A is a low molecular weight compound or a pharmaceutically acceptable salt thereof. In some embodiments the agent suspected or known to reduce activity of TMEM16A is an antibody binding to TMEM16A.

The above explanations with regard to the screening method also apply mutatis mutandis to the agent for use according to the sixth aspect.

According to a seventh aspect, there is provided an agent effective in reducing activity of TMEM6A for use in a method of treating PH in a subject. The subject is generally a subject in need of such treatment.

Treating PH in a subject may involve reducing medial thickness of the pulmonary arteries of a subject suffering from PH. Treating PH in a subject may also involve inhibiting and/or reducing Pulmonary Artery Smooth Muscle (PASMC) proliferation.

In some embodiments the subject is human. In some embodiment the subject is a horse or a pig. In some embodiments the subject is a dog.

The agent effective in reducing activity of TMEM16A is generally a compound or a pharmaceutically acceptable salt thereof. In some embodiments the agent effective in reducing activity of TMEM16A is a low molecular weight compound or a pharmaceutically acceptable salt thereof. In some embodiments the agent effective in reducing activity of TMEM16A is an antibody binding to TMEM16A.

The PH to be treated may be PAH. In some embodiments the PAH is Group 1 PAH. The Group 1 PAH may in some embodiments be idiopathic or primary pulmonary hypertension. As already indicated above, in some embodiments the Group 1 PAH may for example be or include a condition such as familial hypertension and/or another condition defined above. The Group 1 PAH may also be secondary to one or more of the conditions defined above.

In some embodiments the agent effective in reducing activity of TMEM16A is a compound disclosed herein. In some embodiments the agent effective in reducing activity of TMEM16A is an agent, including a compound, that has been identified by a method according to the second aspect or by a method according to the third aspect. In some embodiments the agent effective in reducing activity of TMEM16A is an agent, including a compound, that is identifiable by a method according to the second aspect or by a method according to the third aspect. In some embodiments the agent effective in reducing activity of TMEM6A is an agent, including a compound, that has been identified by a use of a nonhuman animal according to the fourth aspect. In some embodiments the agent effective in reducing activity of TMEM16A is an agent, including a compound, that is identifiable by a use according to the fourth aspect.

In some embodiments the agent effective in reducing activity of TMEM16A is an agent, including a compound, that has been identified by an in vivo method according to the fifth aspect. In some embodiments the agent effective in reducing activity of TMEM16A is an agent, including a compound, that is identifiable by an in vivo method according to the fifth aspect. In some embodiments the agent effective in reducing activity of TMEM16A is an agent, including a compound, that is or has been identified by means of an agent for use according to the sixth aspect. In some embodiments the agent effective in reducing activity of TMEM16A is an agent, including a compound, that is identifiable by means of an agent for use according to the sixth aspect.

The agent effective in reducing activity of TMEM16A may be provided as a component of a pharmaceutical composition.

According to a related eighth aspect, there is provided a method of treating a subject suffering from PH. The method includes administering to a subject in need of such treatment a dose effective against PH of at least one TMEM16A inhibitor or a pharmaceutically acceptable salt thereof.

In some embodiments the PH is PAH.

In some embodiments the subject is human. In some embodiment the subject is a horse or a pig. In some embodiments the subject is a dog.

Where PAH is the PH form to be treated, it may be Group 1 PAH. The Group 1 PAH may in some embodiments be idiopathic or primary pulmonary hypertension. As already indicated above, in some embodiments the Group 1 PAH may for example be or include a condition such as familial hypertension and/or another condition defined above. The Group 1 PAH may also be secondary to one or more of the conditions defined above.

According to a ninth aspect, there is provided a pharmaceutical composition. The pharmaceutical composition contains a TMEM16A inhibitor or a pharmaceutically acceptable salt thereof. The pharmaceutical composition further contains one or more pharmaceutically acceptable carriers. The pharmaceutical composition may also contain one or more further therapeutic, including prophylactic, ingredients. The carrier(s) must be “acceptable” in the sense of being compatible with the other ingredients of the formulation and not deleterious to the recipient thereof.

### BRIEF DESCRIPTION OF THE DRAWINGS

FIG. 1 is a schematic illustration on the effect thought to be underlying the method disclosed herein. Sketched is the effect of TMEM16A upregulation on the resting membrane potential in human pulmonary arterial smooth muscle cells (PASMC) and the pathophysiological consequences thereof. The intracellular Ca²⁺ concentration [Ca²⁺]ᵢ and the function of the pulmonary artery are essentially determined by the membrane potential (Eₘ) of the PASMC. In healthy arteries, TMEM16A channels are present in a low number and are not activated, and due to the negative Eₘ voltage-gated Ca²⁺ channels (VGCC) are closed, so that [Ca²⁺]ᵢ is low. Overexpression and increased activation of TMEM16A channels cause a depolarizing current, raising the Eₘ to around −30 mV. VGCC opening increases [Ca²⁺]ᵢ, leading to contraction of the pulmonary artery and induction of PA wall remodeling including PASMC proliferation.

FIG. 2 illustrates the upregulation of TMEM16A and increased I_{ClCa} in PASMC of idiopathic pulmonary arterial hypertension (IPAH) patients. ACT values were calculated as the difference of TMEM16A and 2 microglobulin expression. FIG. 2A shows the expression of TMEM16A mRNA in laser capture microdissected pulmonary arteries (LCM-PA) of healthy donors and IPAH patients. FIG. 2B shows the expression of TMEM16A mRNA in primary PASMC isolated from healthy donors and IPAH patients. ** p<0.01, unpaired t test.

FIG. 3 depicts a Western blot comparing cell membrane expression of TMEM16A in PASMC of donors and IPAH patients. Membrane and cytosolic protein fractions were separated by cell surface protein biotinylation. The Na⁺-K⁺ ATPase al subunit (NKA α1) and α-tubulin served as loading controls for membrane and cytosolic fractions, respectively.

FIG. 4 depicts whole-cell Ca²⁺ activated Cl⁻ current (I_{ClCa}) traces (FIG. 4A) and normalized current-voltage (I-V) relationships (FIG. 4B) measured with voltage clamp in the PASMC of healthy donors and IPAH patients (n=10 for donors and n=9 for IPAH). ** p<0.01, two-way-ANOVA with Bonferroni post test.

FIG. 5 depicts the effect of 48 hours of hypoxia (HOX) on TMEM16A protein expression compared to a normal oxygen concentration (NOX), analyzed by Western blot. FIG. 5A depicts the membrane fraction of primary PASMC, and FIG. 5B depicts the cytosolic fraction of primary PASMC.

FIG. 6 depicts measurements of whole-cell I_{ClCa}, showing that the proteins detected in FIG. 5 formed functional channels. Measurements of donor PASMC after 48 hrs of hypoxia, compared to cells cultured under normoxic conditions were analysed, n=6 for normoxia and n=8 for hypoxia. *** p<0.001, two-way-ANOVA with Bonferroni post test.

FIG. 7 illustrates that changes in the TMEM16A expression affect the membrane potential. FIG. 7A, B: TMEM16A mRNA expression (A) and total protein level (B) in PASMC treated with either non-silencing control RNA (NS) or TMEM16A siRNA (SI). mRNA expression was studied 48 hours post-transfection and is given as ACT, calculated as the difference of TMEM16A and 2 microglobulin expression. FIG. 7C: I_{ClCa} density in the PASMC of IPAH patients 72 hours after treatment with non-silencing control RNA (NS, n=9) or TMEM16A siRNA (SI, n=7). FIG. 7D: Effect of the TMEM6A inhibitor benzbromarone (BBR, 30 μM) on I_{ClCa} density in the PASMC of IPAH patients (IPAH n=9, IPAH+BBR n=7). FIG. 7E: Membrane potential (Eₘ) values obtained from PASMC of healthy donors and IPAH patients in the absence or presence of the TMEM16A blockers T16Ainh-A01 (“T16”, 10 μM) or benzbromarone (“BBR”, 30 μM). FIG. 7F: Membrane potential (Eₘ) of PASMC 72 hrs after transfection with either non-silencing control RNA (NS) or siRNA against TMEM16A (SI).

FIG. 8 depicts the primers used to assess the expression of Cl⁻ channel and transporter genes or the alternative splicing of the ANO1 gene (Exon “detect” and “missing” primers). Gene name, PubMed Nucleotide accession number used for primer design, forward and reverse primer sequences and the size of the PCR product (in bp) are given. All primers were designed so that the PCR product spans at least one exon-exon junction.

FIG. 9 depicts a vasodilator response in pulmonary arteries to the TMEM16A inhibitor ex vivo. FIG. 9A shows representative traces when the TMEM6A blockers T16Ainh-A01 (“T16”) or benzbromarone (“BBR”) were applied in cumulative doses on U-46619 (30 nM) preconstricted mouse pulmonary artery rings. FIG. 9B shows corresponding dose-response curves (T16Ainh-A01 n=3, benzbromarone n=7).

FIG. 10 shows the effect of benzbromarone in vivo in hypoxia-exposed mice. Under continuous in vivo hemodynamic monitoring, benzbromarone (“BBR”) was administered as a single i.v. bolus of 300 μM in mice exposed to 4 weeks of hypoxia or normoxia (control). FIG. 10A: Pre- and postdrug values in right ventricular systolic pressure (RVSP). FIG. 10B: maximal changes in RVSP. *** p<0.001, two-way ANOVA with Bonferroni post test in FIG. 10A, unpaired t test in FIG. 10B. #p<0.05, unpaired t test in FIG. 10A.

FIG. 11 shows the effect of benzbromarone in vivo in monocrotaline (MCT)-treated rats. Under continuous in vivo hemodynamic monitoring, benzbromarone (“BBR”) was administered as a single i.v. bolus of 300 μM in rats treated with monocrotaline (MCT) or vehicle. FIG. 11A: Pre- and postdrug values in RVSP. FIG. 11B: maximal changes in RVSP. ** p<0.01, *** p<0.001, two-way ANOVA with Bonferroni post test in FIG. 11A, unpaired t test in FIG. 10B. #p<0.05, ###p<0.001, unpaired t test in FIG. 11A.

FIG. 12 illustrates the therapeutic potency of benzbromarone for reverse remodeling, on the basis of mice exposed to hypoxia. FIG. 12A provides a schematic diagram of the experiments using hypoxia-exposed mice. Mice were randomized into three groups. Groups HOX+Veh and HOX+BBR were exposed to 4 weeks of hypoxia, while mice in the control group (n=8) were kept under conditions of normal oxygen concentration. After week 2, subcutaneous slow-release pellets containing either vehicle (HOX+Veh group, n=8) or benzbromarone (HOX+BBR, n=8) were implanted. At week 4, mice underwent hemodynamical analyses and were sacrificed for organ collection, as indicated. FIG. 12B: Assessment of RVSP by means of in vivo hemodynamics. FIG. 12C: Estimation of right ventricular hypertrophy (Fulton-index). FIG. 12D: Analysis of pulmonary arterial remodeling expressed as the percentage change in the number of muscularized and non-muscularized arteries (Control n=3, HOX+Veh n=5, HOX+BBR n=5). ** p<0.01, *** p<0.001, one-way ANOVA with Bonferroni's Multiple Comparison Test was used.

FIG. 13 is a further illustration of the therapeutic potency of benzbromarone for reverse remodeling, based on monocrotaline treated rats. FIG. 13A provides a schematic diagram of the experiments. Rats were randomized into three groups. Groups MCT+Veh and MCT+BBR (n=8 each) were treated with monocrotaline (MCT), while rats in the control group (n=8) received vehicle. Two weeks after MCT treatment, subcutaneous slow-release pellets containing either vehicle (MCT+Veh group) or benzbromarone (MCT+BBR) were implanted. At week 4, all rats were subjected to hemodynamic analyses and organ collection, as indicated. FIG. 13B-D: Echocardiographic assessment of the right ventricular free wall thickness (RVFW Thickness, FIG. 13B), cardiac index (CI, FIG. 13C), and pulmonary artery acceleration time (PAAT, FIG. 13D) at week 4, one day before termination of the experiment. FIG. 13E: RVSP measured by means of in vivo hemodynamics. FIG. 13F: Calculation of right ventricular hypertrophy (Fulton-index). FIG. 13G: Analysis of pulmonary arterial remodeling expressed as the percentage change in the number of muscularized and non-muscularized arteries (Control n=4, MCT+Veh n=7, MCT+BBR n=5). * p<0.05, ** p<0.01, *** p<0.001, one-way ANOVA with Bonferroni's Multiple Comparison Test.

FIG. 14 shows that a loss of TMEM6A function or expression decreases the proliferation of human PASMC. FIG. 14A: PDGF-BB induced proliferation of human donor PASMC, measured by means of thymidine incorporation, in the absence (Veh) or presence of 30 μm benzbromarone (BBR, n=6 in all groups). Changes are expressed as percentage change compared to the untreated controls (Ctrl). FIG. 14B: PDGF-BB induced proliferation of human donor PASMC, measured with thymidine incorporation, 72 hours after treatment with either non-silencing control RNA (NS) or TMEM6A siRNA (SI, n=6 in all groups). Changes are expressed as percentage change compared to the controls treated with non-silencing control RNA only (NS). * p<0.05, ** p<0.01, *** p<0.001, one-way ANOVA with Bonferroni's Multiple Comparison Test.

FIG. 15 shows schematics of a primer design for the assessment of alternative splicing. Primer pairs “detect” were designed to detect the presence of the studied exon. In contrast, primer pairs “missing” resulted in a PCR product only in the absence of the studied exon.

FIG. 16 depicts the vector pQXCIP-Ano1, which is the mammalian retroviral expression vector pQXCIP carrying an Ampicillin resistance gene (-lactamase), pQXCIP-Ano1, which contains TMEM16A, carrying GFP at the N-terminus.

FIG. 17 A depicts the endogenous expression of TMEM16A in PAEC, PASMC and donor lung homogenate (hLH). FIG. 17 B shows immunofluorescence staining of TMEM16A (red) and Von Willebrand factor (vWF; white) in PAEC as well as in DONOR and idiopathic pulmonary arterial hypertension (IPAH) lung tissue; nuclei DAPI staining (blue).

FIG. 18 A Plasmid maps of TMEM16A overexpressing (Ad-Ano1) adenovirus and corresponding control (Ad-Ctrl). Both adenoviruses separately express a fluorescent marker mCherry. FIG. 18 B Representative pictures showing the infection rate of primary human PAECs by Ad-Ano1 and Ad-Ctrl. FIG. 18 C Western Blot showing the overexpression of TMEM16A in PAEC, PASMC and HEK293 cells. As a positive control human lung homogenate (hLH) was used.

FIG. 19 Effect of adenovirus-mediated TMEM16A overexpression on resting membrane potential, proliferation and apoptosis of PAEC and PASMC; *p<0.05, SEM, N=4-9. Overexpression of TMEM16A depolarizes the resting membrane potential of PAEC and PASMC.

FIG. 20 Adenovirus-mediated overexpression of TMEM16A significantly reduces tube-formation of primary human PAEC. Bright-field (BF) and complementary mCherry pictures showing the presence of the overexpressing plasmids; *p<0.05, SEM, N=4.

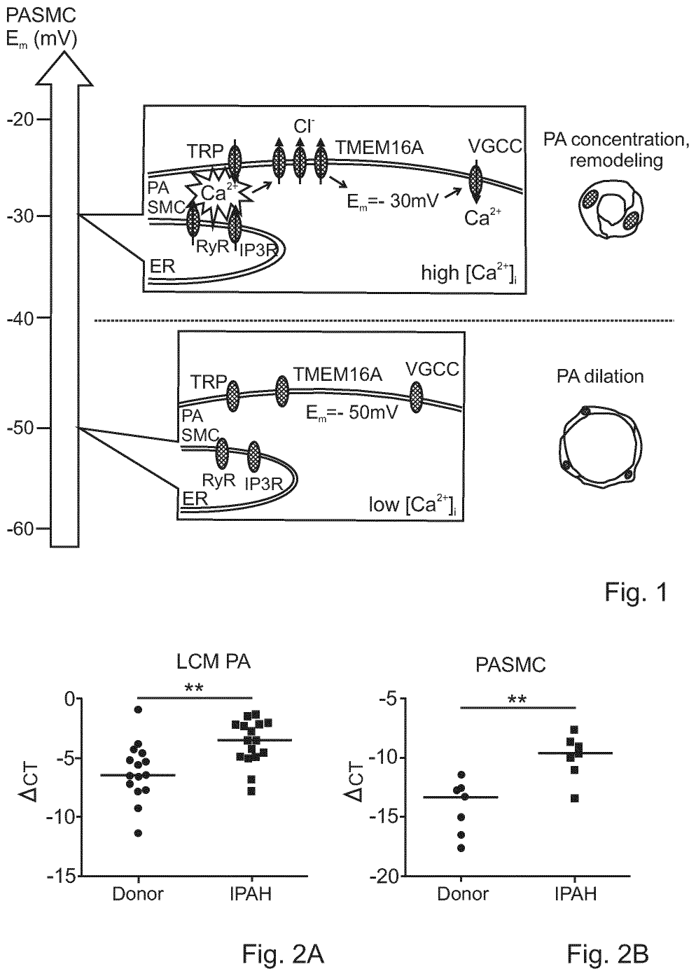

### DETAILED DESCRIPTION

### Definitions

Unless otherwise defined, all other scientific and technical terms used in the description, figures and claims have their ordinary meaning as commonly understood by one of ordinary skill in the art. Although similar or equivalent methods and materials to those described herein can be used in the practice or testing of the binding members, nucleic acids, vectors, host cells, compositions, methods and uses disclosed herein, suitable methods and materials are described below. All publications, patent applications, patents, and other references mentioned herein are incorporated by reference in their entirety. In case of conflict, the present specification, including definitions, will prevail. If two or more documents incorporated by reference include conflicting and/or inconsistent disclosure with respect to each other, then the document having the later effective date shall control. The materials, methods, and examples are illustrative only and not intended to be limiting.

Unless otherwise stated, the following terms used in this document, including the description and claims, have the definitions given below.

The word “about” as used herein refers to a value being within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. The term “about” is also used to indicate that the amount or value in question may be the value designated or some other value that is approximately the same. The phrase is intended to convey that similar values promote equivalent results or effects as described herein. In this context “about” may refer to a range above and/or below of up to 10%. The word “about” refers in some embodiments to a range above and below a certain value that is up to 5%, such as up to 2%, up to 1%, or up to 0.5% above or below that value. In one embodiment “about” refers to a range up to 0.1% above and below a given value.

The term “administering”, as used herein, refers to any mode of transferring, delivering, introducing, or transporting matter such as a compound, e.g. a pharmaceutical compound, or other agent such as an antigen, to a subject. Modes of administration include oral administration, topical contact, intravenous, intraperitoneal, intramuscular, intranasal, or subcutaneous administration (cf. below). Administration “in combination with” further matter such as one or more therapeutic agents includes simultaneous (concurrent) and consecutive administration in any order.

An “agent”, as used herein, refers to any compound or combination of compounds. Typically an agent is a single compound. A respective compound may be a low-molecular weight organic compound or a polymeric compound. An example of a polymeric compound is a protein such as an antibody.

The term “antibody” includes, but is not limited to, an immunoglobulin and a fragment thereof, but it also includes a proteinaceous binding molecule with immunoglobulin-like function. An antibody fragment generally contains an antigen binding or variable region. Examples of (recombinant) antibody fragments are immunoglobulin fragments such as Fab fragments, Fab′ fragments, Fv fragments, single-chain Fv fragments (scFv), diabodies or domain antibodies (Holt, L. J., et al., Trends Biotechnol. (2003), 21, 11, 484-490). An example of a proteinaceous binding molecule with immunoglobulin-like functions is a mutein based on a polypeptide of the lipocalin family (WO 03/029462, Beste et al., Proc. Natl. Acad. Sci. USA (1999) 96, 1898-1903). Lipocalins, such as the bilin binding protein, the human neutrophil gelatinase-associated lipocalin, human Apolipoprotein D or glycodelin, posses natural ligand-binding sites that can be modified so that they bind to selected small protein regions known as haptens. Examples of other proteinaceous binding molecules are the so-called glubodies (see e.g. international patent application WO 96/23879 or Napolitano, E. W., et al., Chemistry & Biology (1996) 3, 5, 359-367), proteins based on the ankyrin scaffold (Mosavi, L. K., et al., Protein Science (2004) 13, 6, 1435-1448) or crystalline scaffold (e.g. internation patent application WO 01/04144), the proteins described in Skerra, J. Mol. Recognit. (2000) 13, 167-187, AdNectins, tetranectins and avimers. Avimers contain so called A-domains that occur as strings of multiple domains in several cell surface receptors (Silverman, J., et al., Nature Biotechnology (2005) 23, 1556-1561). Adnectins, derived from a domain of human fibronectin, contain three loops that can be engineered for immunoglobulin-like binding to targets (Gill, D. S. & Damle, N. K., Current Opinion in Biotechnology (2006) 17, 653-658). Tetranectins, derived from the respective human homotrimeric protein, likewise contain loop regions in a C-type lectin domain that can be engineered for desired binding (ibid.). Peptoids, which can act as protein ligands, are oligo-(N-alkyl) glycines that differ from peptides in that the side chain is connected to the amide nitrogen rather than the carbon atom. Peptoids are typically resistant to proteases and other modifying enzymes and can have a much higher cell permeability than peptides (see e.g. Kwon, Y.-U., and Kodadek, T., J. Am. Chem. Soc. (2007) 129, 1508-1509). A suitable antibody may in some embodiments also be a multispecific antibody that includes several immunoglobulin fragments.

An immunoglobulin or a proteinaceous binding molecule with immunoglobulin-like functions may be PEGylated or hyperglycosylated if desired. In some embodiments a proteinaceous binding molecule with immunoglobulin-like functions is a fusion protein of one of the exemplary proteinaceous binding molecules above and an albumin-binding domain, for instance an albumin-binding domain of streptococcal protein G. In some embodiments a proteinaceous binding molecule with immunoglobulin-like functions is a fusion protein of an immunoglobulin fragment, such as a single-chain diabody, and an immunoglobulin binding domain, for instance a bacterial immunoglobulin binding domain. As an illustrative example, a single-chain diabody may be fused to domain B of staphylococcal protein A as described by Unverdorben et al. (Protein Engineering, Design & Selection [2012] 25, 81-88).

An immunoglobulin may be monoclonal or polyclonal. The term “polyclonal” refers to immunoglobulins that are heterogenous populations of immunoglobulin molecules derived from the sera of animals immunized with an antigen or an antigenic functional derivative thereof. For the production of polyclonal immunoglobulins, one or more of various host animals may be immunized by injection with the antigen. Various adjuvants may be used to increase the immunological response, depending on the host species. “Monoclonal immunoglobulins”, also called “monoclonal antibodies”, are substantially homogenous populations of immunoglobulins to a particular antigen. They may be obtained by any technique which provides for the production of immunoglobulin molecules by continuous cell lines in culture. Monoclonal immunoglobulins may be obtained by methods well known to those skilled in the art (see for example, Köhler et al., Nature (1975) 256, 495-497, and U.S. Pat. No. 4,376,110). An immunoglobulin or immunoglobulin fragment with specific binding affinity only for e.g. TMEM16A can be isolated, enriched, or purified from a prokaryotic or eukaryotic organism. Routine methods known to those skilled in the art enable production of both immunoglobulins or immunoglobulin fragments and proteinaceous binding molecules with immunoglobulin-like functions, in both prokaryotic and eukaryotic organisms.

The term “detect” or “detecting”, as well as the term “determine” or “determining” when used in the context of a signal such as a current, refers to any method that can be used to detect the flow of ions. Detection can be done both on a qualitative and a quantitative level. When used herein in combination with the words “level”, “amount” or “value”, the words “detect”, “detecting”, “determine” or “determining” are understood to generally refer to a quantitative rather than a qualitative level. Accordingly, a method as described herein may include a quantification of a current.

An “effective amount” of a compound is an amount—either as a single dose or as part of a series of doses—which at the dosage regimen applied yields the desired therapeutic effect, i.e., to reach a certain treatment goal. A therapeutically effective amount is generally an amount sufficient to provide a therapeutic benefit in the treatment or management of the relevant pathological condition, or to delay or minimize one or more symptoms associated with the presence of the condition. The dosage will depend on various factors including subject and clinical factors (e.g., age, weight, gender, clinical history of the patient, severity of the disorder and/or response to the treatment), the form of the disorder, e.g. of PH, being treated, the particular composition to be administered, the route of administration, and other factors.

The term “essentially consists of” is understood to allow the presence of additional components in a sample or a composition that do not affect the properties of the sample or a composition. As an illustrative example, a pharmaceutical composition may include excipients if it essentially consists of an active ingredient.

The terms “expressing” and “expression” in reference to a polypeptide are intended to be understood in the ordinary meaning as used in the art. A polypeptide is expressed by a cell via transcription of a nucleic acid into mRNA, followed by translation into an initial polypeptide, which is folded and possibly further processed to a mature polypeptide. The polypeptides discussed in this disclosure are in addition being transported to the surface of the respective cell and integrated into the cell membrane. Hence, the statement that a cell is expressing such a polypeptide indicates that the polypeptide is found on the surface of the cell and implies that the polypeptide has been synthesized by the expression machinery of the respective cell.

With regard to the respective biological process itself, the terms “expression”, “gene expression” or “expressing” refer to the entirety of regulatory pathways converting the information encoded in the nucleic acid sequence of a gene first into messenger RNA (mRNA) and then to a polypeptide. Accordingly, the expression of a gene includes its transcription into a primary hnRNA, the processing of this hnRNA into a mature RNA and the translation of the mRNA sequence into the corresponding amino acid sequence of the polypeptide. In this context, it is also noted that the term “gene product” refers not only to a polypeptide, including e.g. a mature polypeptide (including a splice variant thereof) encoded by that gene and a respective precursor protein where applicable, but also to the respective mRNA, which may be regarded as the “first gene product” during the course of gene expression.

By “fragment” in reference to a polypeptide such as receptor molecule is meant any amino acid sequence present in a corresponding polypeptide, as long as it is shorter than the full length sequence and as long as it is capable of performing the function of interest of the protein—in the case of a calcium-activated chloride channel the diffusion of anions, including a resulting current, through the channel in response to an increase in intracellular Ca²⁺, to cell swelling, and/or to other physiological signals that activate a natural occurring TMEM16A protein.

As used herein, a “heterologous” nucleic acid molecule or a “heterologous” nucleic acid sequence is a nucleic acid molecule and sequence, respectively, that does not occur naturally as part of the genome of the cell in which it is present, or a nucleic acid sequence which is found in a location or locations in the genome that differ from that in which it occurs in nature. Typically, a heterologous nucleic acid molecule and/or sequence carries or is a sequence that is not endogenous to the host cell and has been artificially introduced into the cell. The cell that expresses a heterologous nucleic acid sequence may contain DNA encoding the same or different expression products. A heterologous nucleic acid sequence need not be expressed and may be integrated into the host cell genome or maintained episomally.

The term “isolated” indicates that the cell or cells, or the peptide(s) or nucleic acid molecule(s) has/have been removed from its/their normal physiological environment, e.g. a natural source, or that a peptide or nucleic acid is synthesized. Use of the term “isolated” indicates that a naturally occurring sequence has been removed from its normal cellular (i.e., chromosomal) environment. Thus, the sequence may be in a cell-free solution or placed in a different cellular environment. An isolated cell or isolated cells may for instance be included in a different medium such as an aqueous solution than provided originally, or placed in a different physiological environment. Typically isolated cells, peptides or nucleic acid molecule(s) constitute a higher fraction of the total cells, peptides or nucleic acid molecule(s) present in their environment, e.g. solution/suspension as applicable, than in the environment from which they were taken. An isolated polypeptide or nucleic acid molecule is an oligomer or a polymer of amino acids (2 or more amino acids) or nucleotides coupled to each other, including a polypeptide or nucleic acid molecule that is isolated from a natural source or that is synthesized. The term “isolated” does not imply that the sequence is the only amino acid chain or nucleotide chain present, but that it is essentially free, e.g. about 90-95% pure or more, of e.g. non-amino acid material and/or non-nucleic acid material, respectively, naturally associated with it.

A compound that “modulates the activity of the chloride channel TMEM16A” refers to a compound that alters the activity of TMEM16A so that activity of the channel is different in the presence of the compound than in the absence of the compound. Such compounds may include agonists, inverse agonists and antagonists. The term agonist refers to a substance that activates the chloride channel. The term agonist also refers to a partial agonist. The term inverse agonist is applicable to a protein, typically a receptor, that shows constitutive, also called intrinsic or basal, activity level in the absence of any ligand. For such a protein an agonist increases the activity above its basal level. An inverse agonist may bind to the protein in a manner comparable to an agonist, but it decreases the activity below the basal level.

A compound that “inhibits the activity of the chloride channel TMEM16A” or “inhibits the activity of TMEM16A” refers to a compound that reduces the activity of TMEM16A so that activity of the channel is lower in the presence of the compound than in the absence of the compound. Typically the activity of TMEM16A is reduced when compared to the chloride channel in the absence of any compound with modulating activity.

As used herein, “TMEM16A” means a calcium-activated chloride channel of the anoctamin family, which generally has eight transmembrane segments. In this regard it is well known in the art that the term “chloride channel” does not imply a high selectivity with regard to chloride ions, but that such channels may rather conduct various anions. However, the concentration of chloride in vivo is much higher than the concentration of other anions, rendering the channel in effect a chloride channel. Nevertheless, TMEM6A is known to have a certain selectivity for chloride ions. The function of TMEM6A has so far been unknown. The term “TMEM6A” refers to the protein on the basis of its sequence as such, regardless of modifications such as post-translational modifications. As an illustrative example, the human protein of SwissProt/UniProt accession number Q5XXA6 is known to be glycosylated at asparagine 832, and to be phosphorylated at serines 107 and 196. The term “TMEM6A” refers to any such protein, i.e. regardless of the absence or presence of such modification.

The term “nucleic acid molecule” as used herein refers to any nucleic acid in any possible configuration, such as single stranded, double stranded or a combination thereof. Examples of nucleic acids include for instance DNA molecules, RNA molecules, analogues of the DNA or RNA generated using nucleotide analogues or using nucleic acid chemistry, locked nucleic acid molecules (LNA), protein nucleic acids molecules (PNA), alkylphosphonate and alkylphosphotriester nucleic acid molecules and tecto-RNA molecules (e.g. Liu, B., et al., J. Am. Chem. Soc. (2004) 126, 4076-4077). LNA has a modified RNA backbone with a methylene bridge between C4′ and O2′, providing the respective molecule with a higher duplex stability and nuclease resistance. Alkylphosphonate and alkylphosphotriester nucleic acid molecules can be viewed as a DNA or an RNA molecule, in which phosphate groups of the nucleic acid backbone are neutralized by exchanging the P—OH groups of the phosphate groups in the nucleic acid backbone to an alkyl and to an alkoxy group, respectively. DNA or RNA may be of genomic or synthetic origin and may be single or double stranded. Such nucleic acid can be e.g. mRNA, cRNA, synthetic RNA, genomic DNA, cDNA synthetic DNA, a copolymer of DNA and RNA, oligonucleotides, etc. A respective nucleic acid may furthermore contain non-natural nucleotide analogues and/or be linked to an affinity tag or a label.

Many nucleotide analogues are known and can be used for nucleic acids that are used in the methods described herein. A nucleotide analogue is a nucleotide containing a modification at for instance the base, sugar, or phosphate moieties. As an illustrative example, a substitution of 2′-OH residues of siRNA with 2′F, 2O-Me or 2′H residues is known to improve the in vivo stability of the respective RNA. Modifications at the base moiety may be a natural or a synthetic modification of A, C, G, and T/U, a different purine or pyrimidine base, such as uracil-5-yl, hypoxanthin-9-yl, and 2-aminoadenin-9-yl, as well as a non-purine or a non-pyrimidine nucleotide base. Other nucleotide analogues serve as universal bases. Examples of universal bases include 3-nitropyrrole and 5-nitroindole. Universal bases are able to form a base pair with any other base. Base modifications often can be combined with for example a sugar modification, such as for instance 2′-O-methoxyethyl, e.g. to achieve unique properties such as increased duplex stability.

The term “occurrence of PH” as used in this disclosure includes a condition having one or more characteristics indicative of the presence of PH. As already indicated above, the characteristic of PH is an elevated blood pressure in the pulmonary circulation. The cardinal symptom of every form of PH is progressive exercise dyspnea, often accompanied by fatigue and exhaustion. Symptoms of PH further include, but are not limited to, dizziness, fainting, leg swelling, fatigue, chest pain, palpitations (increased heartbeat rate) and/or pain in the right side of the abdomen. Further details are well known to the practitioner, and can for instance be found in Hoeper et al. (Dtsch Arztebl Int (2017) 114: 73-84).

The term “occurrence of PAH” as used in this disclosure includes a condition having one or more characteristics indicative of the presence of PAH. As explained above, as for any form of PH, the typical characteristic of PAH is a mean pulmonary artery pressure of ≥25 mm Hg. Further characteristics are a pulmonary arterial wedge pressure ≤15 mm Hg and pulmonary vascular resistance of >240 dyn×s×cm⁵. As for PH, further indications of PAH include, but are not limited to, shortness of breath, fatigue, fainting, leg swelling, chest pain, palpitations (increased heartbeat rate) and/or pain in the right side of the abdomen. Yet further indications of PAH include, but are not limited to, poor appetite, light headedness, fainting or syncope, swelling of legs and/or ankles and/or cyanosis.

As used in this document, the expression “pharmaceutically acceptable” refers to those active compounds, materials, compositions, carriers, and/or dosage forms which are, within the scope of sound medical judgment, suitable for use in contact with the tissues of human beings and animals without excessive toxicity, irritation, allergic response, or other problems or complications, commensurate with a reasonable benefit/risk ratio.

The terms “polypeptide” and “protein” are used interchangeably and refer to a polymer of amino acid residues and are not limited to a certain minimum length of the product. Where both terms are used concurrently, this twofold naming accounts for the use of both terms side by side in the art.

The term “predicting the risk” as used in the disclosure refers to assessing the probability that a subject will suffer from PAH in the future. As will be understood by those skilled in the art, such an assessment is usually not intended to be correct for 100% of the subjects to be investigated. The term, however, requires that a prediction can be made for a statistically significant portion of subjects in a proper and correct manner. Whether a portion is statistically significant can be determined by those skilled in the art using various well known statistic evaluation tools, e.g., determination of confidence intervals, p-value determination, Student's t-test, and Mann-Whitney test. Suitable confidence intervals are generally at least 90%, at least 95%, at least 97%, at least 98%, or at least 99%. Suitable p-values are generally 0.1, 0.05, 0.01, 0.005, or 0.0001. In one embodiment of the disclosed methods, the probability envisaged by the present disclosure allows that the prediction of an increased, normal, or decreased risk will be correct for at least 60%, at least 70%, at least 80%, or at least 90% of the subjects of a given cohort or population. Predictions of risk in a disclosed method relates to predicting whether or not there is an increased risk for PAH compared to the average risk for developing PAH in a population of subjects rather than giving a precise probability for the risk.

In this regard the term “prognosis”, commonplace and well-understood in medical and clinical practice, refers to a forecast, a prediction, an advance declaration, or foretelling of the probability of occurrence of a disease state or condition in a subject not (yet) having the respective disease state or condition. In the context of the present invention prognosis refers to the forecast or prediction of the probability as to whether a subject will or will not suffer from PAH.

The term “preventing” in the medical/physiological context, i.e. in the context of a physiological state, refers to decreasing the probability that an organism contracts or develops an abnormal condition.

The term “purified” is understood to be a relative indication in comparison to the original environment of the cell, thereby representing an indication that the cell is relatively purer than in the natural environment. It therefore includes, but does not only refer to, an absolute value in the sense of absolute purity from other cells (such as a homogeneous cell population). Compared to the natural level, the level after purifying the cell will generally be at least 2-5 fold greater (e.g., in terms of cells/ml). Purification of at least one order of magnitude, such as about two or three orders, including for example about four or five orders of magnitude is expressly contemplated. It may be desired to obtain the cell at least essentially free of contamination, in particular free of other cells, at a functionally significant level, for example about 95%, about 95%, or 99% pure. With regard to a nucleic acid, peptide or a protein, the above applies mutatis mutandis. In this case purifying the nucleic acid, peptide or protein will for instance generally be at least 2-5 fold greater (e.g., in terms of mg/ml).

The word “recombinant” is used in this document to describe a nucleic acid molecule that, by virtue of its origin, manipulation, or both is not associated with all or a portion of the nucleic acid molecule with which it is associated in nature. Generally a recombinant nucleic acid molecule includes a sequence which does not naturally occur in the respective wildtype organism or cell. Typically a recombinant nucleic acid molecule is obtained by genetic engineering, usually constructed outside of a cell. Generally a recombinant nucleic acid molecule is substantially identical and/or substantial complementary to at least a portion of the corresponding nucleic acid molecule occurring in nature. A recombinant nucleic acid molecule may be of any origin, such as genomic, cDNA, mammalian, bacterial, viral, semisynthetic or synthetic origin. The term “recombinant” as used with respect to a protein/polypeptide means a polypeptide produced by expression of a recombinant polynucleotide.

The term “reducing the risk”, as used in this document, means to lower the likelihood or probability of a disease state or condition, e.g., PH such as PAH, from occurring in a subject, especially when the subject is predisposed to such or at risk of contracting a disease state or condition, e.g., PH.

The term “subject” as used herein, also addressed as an individual, refers to an animal, generally a mammal. A subject may be a mammalian species such as a cattle or a goat. The subject may also be a sheep. In some embodiments the subject is a horse. The subject may also be a dog or a cat. In some embodiments the subject is a ferret or a chinchilla. In some embodiments the subject is a pig. The subject may also be a monkey, a rabbit, a mouse, a rat, a Guinea pig, a hamster, an ape or a human.

The terms “treatment” and “treating” as used herein, refer to a prophylactic or preventative measure having a therapeutic effect and preventing, slowing down (lessen), or at least partially alleviating or abrogating an abnormal, including pathologic, condition in the organism of a subject. Those in need of treatment include those already with the disorder as well as those prone to having the disorder or those in whom the disorder is to be prevented (prophylaxis). Generally a treatment reduces, stabilizes, or inhibits progression of a symptom that is associated with the presence and/or progression of a disease or pathological condition. The term “therapeutic effect” refers to the inhibition or activation of factors causing or contributing to the abnormal condition. A therapeutic effect relieves to some extent one or more of the symptoms of an abnormal condition or disease. The term “abnormal condition” refers to a function in the cells or tissues of an organism that deviates from their normal functions in that organism. An abnormal condition can inter alia relate to cell proliferation, cell differentiation, or cell survival.

The term “variant” as used herein can refer to a nucleotide sequence in which the sequence differs from the sequence most prevalent in a population, for example by one nucleotide, in the case of the point mutations described herein. For example, some variations or substitutions in the nucleotide sequence encoding a TMEM16A protein can alter a codon so that a different amino acid is encoded resulting in a variant polypeptide. The term “variant” can also refer to a polypeptide in which the sequence differs from a given sequence as explained further below. A variant may for example be a polypeptide in which the sequence differs from the sequence most prevalent in a population. A polypeptide sequence can for instance differ at a position that does not change the amino acid sequence of the encoded polypeptide, i.e. a conserved change. Variant polypeptides can be encoded by a mutated TMEM16A sequence.

The terms “comprising”, “including,” containing”, “having” etc. shall be read expansively or open-ended and without limitation. Singular forms such as “a”, “an” or “the” include plural references unless the context clearly indicates otherwise. Thus, for example, reference to a “vector” includes a single vector as well as a plurality of vectors, either the same—e.g. the same operon—or different. Likewise reference to “cell” includes a single cell as well as a plurality of cells. Unless otherwise indicated, the term “at least” preceding a series of elements is to be understood to refer to every element in the series. The terms “at least one” and “at least one of” include for example, one, two, three, four, or five or more elements. It is furthermore understood that slight variations above and below a stated range can be used to achieve substantially the same results as a value within the range. Also, unless indicated otherwise, the disclosure of ranges is intended as a continuous range including every value between the minimum and maximum values.

The scope and meaning of any use of a term will be apparent from the specific context in which the term is used. Certain further definitions for selected terms used throughout this document are given in the appropriate context of the detailed description, as applicable. Unless otherwise defined, all other scientific and technical terms used in the description, figures and claims have their ordinary meaning as commonly understood by one of ordinary skill in the art.

### Action of Compounds on the TMEM16A Channel

The mean arterial pressure is determined by the cardiac output and the peripheral resistance. Besides cerebral control and the sympathetic nervous system, a varity of factors control the vascular tone, such as the catecholamines adrenalin and noradrenalin, the renin-angiotensin-aldosterone system, the vasculary nitric oxide (NO) system, natriuretic peptides, phosphodiesterase 5 (PDE5) or endothelin peptides. Vasoconstrictors such as thromboxane and vascular endothelial growth factor (VEGF) likewise influence arterial pressure, and an increased synthesis of them has been found in PAH.

The opening of cellular calcium channels allows the influx of calcium ions, which causes a vasoconstriction. In smooth muscle cells and in the heart, voltage-dependent L-type calcium channels allow a calcium influx and thereby a contraction. Cellular potassium channels largely determine the resting potential of a cell such as a smooth muscle cell. The opening of potassium channels shifts the resting potential toward the equilibrium potential of potassium, thereby rendering the potential more negative, called hyperpolarization. Thereby the influx of calcium via voltage-dependent calcium channels is being reduced.

The pathophysiologic mechanism of PAH is known to involve several signaling pathways, including depolarization and Ca²⁺ overload of the pulmonary arterial smooth muscle cells (PASMC). Both the membrane depolarization and the Ca²⁺ overload have so far been thought to result from altered expression and function of different cation channels such as potassium channels and cation transporters, as well as Ca²⁺ handling proteins. For example, decreased gene expression or loss-of-function mutation of voltage-gated (Yuan, J X, et al., Circulation (1998) 98, 1400-1406; Yuan, J X, et al., Lancet (1998) 351, 726-727) and two-pore domain potassium channels (Antigny, F, et al., Circulation (2016) 133, 1371-1385; Ma, L, et al., N. Engl. J. Med. (2013) 369, 351-361), increased expression of nonselective cation channels (Xia, Y, et al., Hypertension (2014) 63, 173-180; Yu, Y, et al., Proc. Natl. Acad. Sci. U.S.A. (2004) 101, 13861-13866; Zhang, M F, et al., Sheng Li Xue Bao (2010) 62, 55-62) and the Na⁺/Ca²⁺ exchanger (Zhang, S, et al., Am. J. Physiol. Cell. Physiol. (2007) 292, C2297-305) have been reported to occur in IPAH.

The present inventors have surprisingly found that the calcium-activated chloride channel TMEM16A is overexpressed in PH, including PAH. In further studies, the results of some are disclosed herein, the inventors have identified the increased TMEM16A activity as an important pathologic mechanism underlying vasoconstriction and remodeling of pulmonary arteries. The methods and used disclosed herein were then developed when the inventors inter alia further found that chronic benzbromarone treatment reverses the development of PH in animal models.

While the presence of Ca²⁺ activated Cl⁻ currents in smooth muscle cells has been known for a long time, anion channels have so far not been taken into consideration in searching for factors involved in the disease. Nevertheless, reports that identify the encoding genes (Yang, Y D, et al. Nature (2008) 455, 1210-1215; Caputo, A, et al., Science (2008) 322, 590-594; Schroeder, B C, et al., Cell (2008) 134, 1019-1029) as well as selective blockers of these channels (inter alia Davis, A J, et al., Br. J. Pharmacol. (2013) 168, 773-784; Huang, F. et al., Proc. Natl. Acad. Sci. U.S.A. (2012) 109, 16354-16359) have provided tools that can be used in the context of the methods disclosed herein. The methods and use disclosed herein, and the underlying findings by the inventors explain a previous observation on changes in the expression of TMEM16A in pulmonary arteries in in the monocrotaline (MCT)-induced PH model in the rat (PH. Forrest, A S, et al., Am. J. Physiol. Cell. Physiol. (2012) 303, C1229-1243).

Chloride channels conduct outward rectifier currents in vascular smooth muscle cells causing depolarizing oscillations in the resting membrane resulting in vasoconstriction. Chronic activation may cause cell depolarisation, vasoconstriction and vascular remodelling. The Ca²⁺ activated Cl⁻ channel TMEM16A is active at physiological resting membrane potential in human pulmonary arterial smooth muscle cells (approx. −50 mV). Since the intracellular Cl⁻ concentration of a pulmonary arterial smooth muscle cell is relatively high (approx. 45 mM), opening of TMEM6A channels results in a Cl⁻ efflux leading to depolarization of the smooth muscle cell and subsequent Ca²⁺ influx. TMEM16A is expressed in the pulmonary artery of several species including humans (Manoury, B, et al., Journal of Physiology-London (2010) 588).

### TMEM16A and Nucleic Acid Sequences Encoding the Same

TMEM16A is a protein that may be the human anoctamin protein of SwissProt/UniProt accession number W6JLH6, version 16 of 15 Mar. 2017 (version 1 of the sequence). The protein may also be the human Anoctamin-1 protein, also known as transmembrane protein 16A, oral cancer overexpressed protein 2 or tumour-amplified and overexpressed sequence 2, which has SwissProt/UniProt accession number Q5XXA6, version 111 of 15 Feb. 2017 (version 1 of the sequence). The human Anoctamin-1 protein depends on ATP and calmodulin for its activation. Human Anoctamin-1 of SwissProt/UniProt accession number Q5XXA6 exists in different isoforms, formed by alternative splicing. Any such isoform falls under the term “TMEM16A”. Version 111, dated 15 Feb. 2017, of the SwissProt/UniProt entry names three isoforms, called isoforms 1 to 3, and provided with identifiers Q5XXA6-1 to Q5XXA6-3. Isoform 1 has a total of 986 amino acids, isoform 2 a total of 840 amino acids, and isoform 3 a total of 642 amino acids.

TMEM16A may also be the human chloride channel of SwissProt/UniProt accession number A0A0G2QSF1, version 9 of 12 Apr. 2017 (version 1 of the sequence). In some embodiments TMEM6A is the human protein encoded by the sequence of Genbank accession number AY728143, version 1 as of 28 Sep. 2004. In some embodiments TMEM6A is the human protein encoded by the sequence of Genbank accession number NR_030691, version 1 as of 23 Apr. 2017. TMEM16A may in some embodiments be the human protein encoded by the sequence of Genbank accession number HQ418153, version 1 as of 25 Jul. 2016. In some embodiments TMEM16A is the human protein encoded by the sequence of Genbank accession number NM_018043, version 5 as of 23 Apr. 2017. TMEM16A may in some embodiments be the human protein encoded by the sequence of Genbank accession number XM_011545121, version 2 as of 6 Jun. 2016. TMEM16A may in some embodiments be the human protein encoded by transcript variant X1 of the sequence of Genbank accession number XM_011545121, version 2 as of 6 Jun. 2016. In some embodiments TMEM16A is the human protein encoded by transcript variant X2 of the sequence of Genbank accession number XM_011545123, version 2 as of 6 Jun. 2016. In some embodiments TMEM6A is the human protein encoded by transcript variant X3 of the sequence of Genbank accession number XM_011545124, version 2 as of 6 Jun. 2016. TMEM16A may in some embodiments be the human protein encoded by transcript variant X4 of the sequence of Genbank accession number XM_017017956, version 1 as of 6 Jun. 2016. In some embodiments TMEM16A is the human protein encoded by transcript variant X5 of the sequence of Genbank accession number XM_011545125, version 2 as of 6 Jun. 2016. In some embodiments TMEM16A is the human protein encoded by transcript variant X6 of the sequence of Genbank accession number XM_017017957, version 1 as of 6 Jun. 2016. TMEM16A may in some embodiments be the human protein encoded by transcript variant X7 of the sequence of Genbank accession number XM_011545126, version 2 as of 6 Jun. 2016. In some embodiments TMEM16A is the human protein encoded by transcript variant X8 of the sequence of Genbank accession number XM_011545127, version 2 as of 6 Jun. 2016. TMEM16A may in some embodiments be the human protein encoded by transcript variant X9 of the sequence of Genbank accession number XM_011545128, version 2 as of 6 Jun. 2016. In some embodiments TMEM6A is the human protein encoded by transcript variant X10 of the sequence of Genbank accession number XM_006718602, version 2 as of 6 Jun. 2016. In some embodiments TMEM16A is the human protein encoded by transcript variant X11 of the sequence of Genbank accession number XM_011545129, version 2 as of 6 Jun. 2016. TMEM16A may in some embodiments be the human protein encoded by transcript variant X12 of the sequence of Genbank accession number XM_006718604, version 2 as of 6 Jun. 2016. In some embodiments TMEM16A is the human protein encoded by transcript variant X13 of the sequence of Genbank accession number XM_006718605, version 2 as of 6 Jun. 2016. TMEM6A may in some embodiments be the human protein encoded by transcript variant X14 of the sequence of Genbank accession number XM_011545131, version 2 as of 6 Jun. 2016.

TMEM6A may also be the human chloride channel encoded by the sequence of Genbank accession number AB845669, version 1 as of 5 Feb. 2014. In some embodiments TMEM16A may be a human protein encoded by a sequence comprising Genbank accession number KC577595, version 1 as of 21 Jun. 2015. In some embodiments TMEM16A is a Rhesus macaque protein encoded by the sequence of Genbank accession number BV448621, version 1 as of 30 Mar. 2005. In some embodiments TMEM16A is the Bonobo (pygmy chimpanzee, Pan paniscus) protein encoded by transcript variant X1 of the sequence of Genbank accession number XM_008954212, version 1 as of 30 Sep. 2015. TMEM16A may in some embodiments be the Bonobo protein encoded by transcript variant X2 of the sequence of Genbank accession number XM_008954213, version 1 as of 30 Sep. 2015. In some embodiments TMEM16A may be the Bonobo protein encoded by transcript variant X3 of the sequence of Genbank accession number XM_008954214, version 1 as of 30 Sep. 2015.

TMEM16A may in some embodiments be the human protein encoded by SEQ ID NO: 2 of US patent application US 2013/323252, Genbank accession number HK730470, version 1 as of 10 Feb. 2016. In some embodiments TMEM16A is the Macaca nemestrina protein encoded by transcript variant X1 of the sequence of Genbank accession number XM_011721179, or by transcript variant X2 of the sequence of Genbank accession number XM_011721180, both version 1 as of 30 Mar. 2015. In some embodiments TMEM6A is the Macaca nemestrina protein encoded by transcript variant X3 of the sequence of Genbank accession number XM_011721181, or by transcript variant X4 of the sequence of Genbank accession number XM_011721182, again both version 1 as of 30 Mar. 2015. TMEM16A may also be the Macaca nemestrina protein encoded by transcript variant X5 of the sequence of Genbank accession number XM_011721183, or by transcript variant X6 of the sequence of Genbank accession number XM_011721184, again both version 1 as of 30 Mar. 2015. In some embodiments TMEM16A is the Macaca nemestrina protein encoded by transcript variant X7 of the sequence of Genbank accession number XM_011721185, or by transcript variant X8 of the sequence of Genbank accession number XM_011721186, both version 1 as of 30 Mar. 2015. In some embodiments TMEM16A is the Macaca nemestrina protein encoded by transcript variant X9 of the sequence of Genbank accession number XM_011721188, or by transcript variant X10 of the sequence of Genbank accession number XM_011721189, both version 1 as of 30 Mar. 2015.

In some embodiments TMEM16A is the protein of the old world monkey sooty mangabey (Cercocebus atys) encoded by transcript variant X1 of the sequence of Genbank accession number XM_012041318, or by transcript variant X2 of the sequence of Genbank accession number XM_012041319, both version 1 as of 30 Mar. 2015. TMEM16A may also be the Cercocebus atys protein encoded by transcript variant X3 of the sequence of Genbank accession number XM_012041320, or by transcript variant X4 of the sequence of Genbank accession number XM_012041321, again both version 1 as of 30 Mar. 2015. In some embodiments TMEM16A is the Cercocebus atys protein encoded by transcript variant X5 of the sequence of Genbank accession number XM_012041322, or by transcript variant X6 of the sequence of Genbank accession number XM_012041324, both version 1 as of 30 Mar. 2015. In some embodiments TMEM16A is the Cercocebus atys protein encoded by transcript variant X7 of the sequence of Genbank accession number XM_012041325, version 1 as of 30 Mar. 2015.

TMEM16A may also be the murine protein Anoctamin-1 of SwissProt/UniProt accession number Q8BHY3, version 114 of 15 Feb. 2017 (version 2 of the sequence). Murine Anoctamin-1 of SwissProt/UniProt accession number Q8BHY3 exists in different isoforms, formed by alternative splicing. Again, any such isoform falls under the term “TMEM16A”. Version 115, dated 15 Feb. 2017, of the SwissProt/UniProt entry names two isoforms, called isoforms 1 and 2, and provided with identifiers Q8BHY3-1 and Q8BHY3-2. Isoform 1 has a total of 960 amino acids, and isoform 2 a total of 956 amino acids.

In some embodiments TMEM16A is the rat protein Anoctamin of SwissProt/UniProt accession number D4A915, version 63 of 12 Apr. 2017 (version 1 of the sequence). In some embodiments TMEM16A is the protein Anoctamin from Xenopus laevis with SwissProt/UniProt accession number B5SVV6, version 36 of 15 Mar. 2017 (version 1 of the sequence).

TMEM16A may also be a guinea pig protein encoded by the gene of NCBI Gene ID 100529098 as of 8 Apr. 2017, Genbank accession number NT_176377, version 1 as of 14 Jul. 2015. The gene yields a number of isoforms on the RNA level, Gene ID 100529098 as of 8 Apr. 2017 names anoctamin-1 isoforms X1 to X8. TMEM6A may also be a porcine protein encoded by the gene of Genbank accession number NC_010444.3 as of 11 Sep. 2015. The gene yields different isoforms on the RNA level, NCBI Gene ID 100518411 as of 3 Apr. 2017 names anoctamin-1 isoforms X1 and X2. In some embodiments TMEM6A is the porcine protein encoded by transcript variant X1 of the sequence of Genbank accession number KJ416137, or by transcript variant X2 of the sequence of Genbank accession number KJ416138, both version 1 as of 15 Mar. 2014. In some embodiments TMEM6A is the porcine protein encoded by a sequence that includes the sequence of Genbank accession number KJ416136, version 1 as of 15 Mar. 2014.

In some embodiments TMEM16A is a horse protein encoded by the gene of Genbank accession number NC_009155 as of 20 Nov. 2015. The gene yields different isoforms on the RNA level, NCBI Gene ID 100061836 as of 2 Apr. 2017 names anoctamin-1 isoforms X1 and X2. TMEM16A may also be a dog protein encoded by the gene of Genbank accession number NC_006600 as of 17 Sep. 2015. In some embodiments TMEM16A is a chicken protein encoded by the gene of Genbank accession number NC_006092 as of 4 Jan. 2016. TMEM16A may also be a bovine protein encoded by the gene of AC_000186 as of 26 Jan. 2016. The bovine gene yields different isoforms on the RNA level, NCBI Gene ID 532126 as of 2 Apr. 2017 names anoctamin-1 isoforms X1 to X3.

In some embodiments TMEM16A may be a functional fragment of a TMEM16A protein as expressed in nature. The respective TMEM16A protein as expressed in nature may be any calcium-activated anoctamin chloride channel, for example one of the above named proteins. Such a fragment is generally a fragment of a continuous length. Typically a functional fragment of a TMEM16A protein is encoded by a nucleic acid sequence of at least about 600 bases, such as at least about 600 bases. A functional fragment is capable of forming a calcium-activated chloride channel (CaCC). A functional fragment allows the diffusion of anions through the channel in response to an increase in intracellular Ca², to cell swelling, and/or to other physiological signals that activate a natural occurring TMEM6A protein. A functional fragment also allows the formation of a resulting current. Typically a functional fragment has the 8-transmembrane topology known for the full-length protein TMEM6A.

A functional fragment has in some embodiments a length of 900 amino acids or less. In some embodiments a functional fragment has a length of 800 amino acids or less. A functional fragment has in some embodiments a length of 600 amino acids or less, including 550 amino acids or less. In some embodiments a functional fragment has a length of 650 amino acids or more. In some embodiments a functional fragment has a length of 750 amino acids or more, including 850 amino acids or more.

In some embodiments TMEM16A is a variant of a naturally occurring TMEM16A protein. For example, it may be negligible to exchange certain amino acid residues that are nor critical for allowing the diffusion of anions through the channel in response to an increase in intracellular Ca²⁺ or to cell swelling. It may also be desired to modify a TMEM16A protein at one or more positions for detection purposes. A respective variant is a protein that contains an amino acid sequence that has at least about 98% sequence identity to a naturally existing TMEM16A protein. In some embodiments a respective variant contains an amino acid sequence that has at least about 99% sequence identity to a naturally existing TMEM16A protein. A variant of the TMEM16A protein is typically functional in that it allows the diffusion of anions through the channel in response to an increase in intracellular Ca²⁺, to cell swelling, and/or to other physiological signals that activate a natural occurring TMEM16A protein. Like a functional fragment, a variant also allows the formation of a resulting current. A variant also has the 8-transmembrane topology as a naturally occurring TMEM16A protein.

Typically the difference from a naturally occurring TMEM16A protein is a substitution. In some embodiments the difference from a naturally occurring TMEM6A protein is a deletion. A variant of a naturally occurring TMEM6A protein may be a gene obtained from the expression of a gene sequence altered by sitespecific mutagenesis.

Variants of naturally occurring TMEM6A protein may be prepared by protein and/or chemical engineering, introducing appropriate modifications into the nucleic acid sequence encoding the polypeptide, or by protein/peptide synthesis. A variant may be obtained by any combination(s) of one or more deletions, substitutions, additions and insertions to nucleic acid and/or the amino acid sequence, provided that the obtained polypeptide defines a functional TMEM16A channel. In some embodiments a variant of a polypeptide provided herein differs from a particular sequence of a polypeptide provided herein by up to five substitutions. A substitution in an amino acid sequence of a polypeptide provided herein may be a conservative substitution. Examples of conservative substitutions include:
1. Substituting alanine (A) by valine (V);
2. Substituting arginine (R) by lysine (K);
3. Substituting asparagine (N) by glutamine (Q);
4. Substituting aspartic acid (D) by glutamic acid (E);
5. Substituting cysteine (C) by serine (S);
6. Substituting glutamic acid (E) by aspartic acid (D);
7. Substituting glycine (G) by alanine (A);
8. Substituting histidine (H) by arginine (R) or lysine (K);
9. Substituting isoleucine (I) by leucine (L);
10. Substituting methionine (M) by leucine (L);
11. Substituting phenylalanine (F) by tyrosine (Y);
12. Substituting proline (P) by alanine (A);
13. Substituting serine (S) by threonine (T);
14. Substituting tryptophan (W) by tyrosine (Y);
15. Substituting phenylalanine (F) by tryptophan (W); and/or
16. Substituting valine (V) by leucine (L) and vice versa.

A variant of a naturally occurring TMEM16A protein may include one or more, such as two or three of such conservative substitutions. In some embodiments a polypeptide according to this disclosure includes a sequence that has four or more conservative substitutions in comparison to a naturally occurring TMEM16A protein. In some embodiments a variant includes a sequence that has five or more, such as six or more conservative substitutions.

Non-conservative substitutions may lead to more substantial changes, e.g., with respect to the charge, dipole moment, size, hydrophilicity, hydrophobicity or conformation of the polypeptide. In some embodiments the polypeptide includes one or more, such as two non-conservative substitutions. In some embodiments a variant of a naturally occurring TMEM16A protein includes three or four non-conservative substitutions. The variant may also include five or more, e.g. six, or seven or more of such non-conservative substitutions.

### Suitable Compounds inhibiting TMEM16A

The activity of TMEM16A is inhibited by chloride channel inhibitors such as the diphenylcarboxylate compound niflumic acid (NFA). Niflumic acid is also an inhibitor of cyclooxygenase-2, and is used in the treatment of joint and muscular pain. A further diphenylcarboxylate that is a chloride channel inhibitor inhibiting the activity of TMEM16A, is flufenamic acid (FFA, N-(3-[trifluoromethyl]phenyl)anthranilic acid), also an inhibitor of cyclooxygenase-2. Yet another suitable diphenylcarboxylate, likewise an inhibitor of cyclooxygenase-2, is meclofenamic acid (MFA, 2-[(2,6-dichloro-3-methylphenyl)amino]benzoic acid).

Another inhibitor of TMEM16A activity is (R*,S*)-(±)-α-2-Piperidinyl-2,8-bis(trifluoro-methyl)-4-quinolinemethanol, called Mefloquine. This compound is used in the treatment, including the prevention, of malaria. It is on the WHO's List of Essential Medicines.

Further TMEM16A inhibitors are the antimicrobial agents dichlorophen (2,2′-Methylenebis(4-chlorophenol) and hexachlorophene (2,2′-Methylenebis(3,4,6-trichlorphenol)).

Another TMEM16A inhibitor is the antifungal compound miconazole ((RS)-1-[2,4-Dichlor-β-(2,4-dichlorbenzyloxy)-phenethyl]imidazol). Yet another TMEM16A inhibitor is 5-hydroxy-2-methyl-naphthalene-1,4-dione, called plumbagin. The compound is thought to be a toxin, and has been isolated from the plant genus Plumbago.

Further inhibitors of the activity TMEM16A have been disclosed by Oh et al. (Molecular Pharmacology (2013) 84, 5, 726-735), incorporated herein by reference for all purposes in its entirety. In case of conflict, the present document, including definitions, will prevail. Oh et al. have identified compounds of the following structure as inhibitors of TMEM16A:

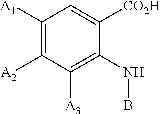

In this formula one of A₁, A₂ and A₃ is selected from a nitro group and a trifluoromethyl group, while the other two positions of A₁, A₂ and A₃ are generally hydrogen. B is a phenyl or a naphthyl group carrying one substituent selected from —OMe, halogen and —CF₃. In some embodiments a phenyl group carries such a substituent at the para position. In some embodiments a naphthyl group carries such a substituent at the 4-position, and is bonded to the amino group of the above formula at its 1- or 2-position. The most potent blocker of TMEM16A identified by Oh et al. is N-((4-methoxy)-2-naphthyl)-5-nitroanthranilic acid, called MONNA.

Yet a further inhibitor of TMEM6A activity is 4,4′-diisothiocyanatostilbene-2,2′-disulfonic acid (DIDS), mainly known as an anion exchange inhibitor, e.g. an inhibitor of the chloride-bicarbonate exchanger.

5-nitro-2-(3-phenylpropylamino)-benzoic acid (NPPB) is another example of an inhibitor of TMEM6A activity. The compound is also known for its action as an inhibitor of volume-regulated anion channels.

Benzbromarone ((2-Ethyl-3-benzofuranyl)-(3,5-dibrom-4-hydroxyphenyl)ketone) is yet a further example of a TMEM16A inhibitor. This compound is also a non-competitive inhibitor of xanthine oxidase and used in the treatment of gout.

A compound developed for the treatment of Alzheimer's disease and other cognitive defects, idebenone (2-(10-hydroxydecyl)-5,6-dimethoxy-3-methyl-cyclohexa-2,5-dien-1,4-dion) is also a suitable inhibitor of TMEM16A activity. Idebenone is a synthetic analogue of coenzyme Q10.

Yet a further inhibitor of TMEM6A activity is tannic acid (2,3-dihydroxy-5-({[(2R,3R, 4S,5R,6R)-3,4,5,6-tetrakis({3,4-dihydroxy-5-[(3,4,5-trihydroxyphenyl)carbonyloxy]phenyl}carbonyl-oxy)oxan-2-yl]methoxy}carbonyl)phenyl 3,4,5-trihydroxybenzoate, also known as acidum tannicum or digallic acid.

The compound CaCCinh-A01 (6-(1,1-Dimethylethyl)-2-[(2-furanylcarbonyl)amino]-4,5,6,7-tetrahydrobenzo[b]thiophene-3-carboxylic acid) is yet a further example of an inhibitor of TMEM16A activity. The compound T16Ainh-A01 (2-[(5-Ethyl-1,6-dihydro-4-methyl-6-oxo-2-pyrimidinyl)thio]-N-[4-(4-methoxyphenyl)-2-thiazolyl]-acetamide) is another example of an inhibitor of TMEM16A activity.

The generation of antibodies to a certain protein is well known in the art, and standard techniques can be employed to obtain an antibody directed against TMEM16A.

Illustrative examples of an antibody directed against TMEM16A have been disclosed in US patent application US 2013/323252. Some antibodies disclosed therein specifically bind to a peptide sequence of KLIRYLKLKQ. Some antibodies disclosed in US 2013/323252 specifically bind to a peptide sequence of RYKDYREPPWS. US 2013/323252 is incorporated herein by reference for all purposes in its entirety including all tables, figures, and claims. In case of conflict, the present document, including definitions, will prevail.

The activity of TMEM16A is also inhibited by the cystic fibrosis transmembrane conductance regulator, abbreviated the CFTR protein. The CFTR protein is also a chloride channel and as such a membrane protein, it is an ATP-gated anion channel. CFTR has been found in smooth muscle cells. In this regard, a compound that stimulates the CFTR protein will typically act as an inhibitor of TMEM16A.

Any agent, e.g. one of the above compounds, an anti-TMEM16A antibody, or a compound identified by a method disclosed herein, may be administered in combination with any other compound suitable for alleviating the symptoms of PH, or for treating PH. Illustrative examples of such compounds are e.g. a diuretic or an anticoagulant. In particular where the subject suffers from PAH, compounds that may be administered in combination with an agent disclosed herein or identified by a method disclosed herein, include an Endothelin receptor antagonist such as Ambrisentan, Bosentan or Macitentan, and a PDE5 inhibitor, e.g. Sildenafil or Tadalafil. Further compounds suitable for such a combination include, without being limited thereto, a stimulator of soluble guanylate cyclase, e.g. Riociguat, or a prostacyclin analog such as Epoprostenol, Iloprost, or Treprostinil. Another example of a compound suitable for a combination is a prostacycline receptor agonist such as Selexipag.

A further example of a compound suitable for a combination with an agent disclosed herein or identified by a method disclosed herein is a calcium antagonist, e.g. a Dihydropyridine compound such as Nifedipine, Efonidipine or Nitrendipine, a phenylalkylamine such as Verapamil, Gallopamil or Fendiline, a Benzothiazepine such as Diltiazem, a Gabapentinoids, such as gabapentin and pregabalin, or any other calcium antagonist such as mibefradil, flunarizine, fluspirilene, fendiline, or Ziconotide. A combination with a calcium antagonist may in particular be suitable in a case where the subject suffers from IPAH or drug-associated PAH. In line with current practice, oxygen therapy can be applied in parallel to administration of an agent disclosed herein, or identified by a method disclosed herein, in case of occurrence of manifest hypoxemia with arterial pO2<60 mm Hg.

### Identifying Further Suitable Compounds

In a method of identifying a compound capable of modulating activity of a TMEM16A channel a variety of techniques known in the art can be employed. Such techniques typically allow the formation of a detectable electrophysiological response to a known agonist. The respective response may be detected in the form of measurements of ionic currents. Automated electrophysiology instruments using planar arrays are commercially available to perform this task. Since an ion flux through a channel affects membrane potential, the respective response may also be detected using an electrophysiological method. As an example, conventional patch-clamp electrophysiology measurements can be employed. Changes in membrane potential may also be detected using a dye system, for example using a fluorescent dye. A fluorescent plate reader with kinetic capabilities may for instance be employed in this regard. 96-, 384-, or 1536-well plate formats may be used. The respective response may also be detected on the basis of a change in ion concentration on one side of a membrane. This may be achieved by an electromagnetic signal, for instance by means of a sensor dye that indicates the presence and/or absence of certain metal ions. Again, 96-, 384-, or 1536-well plates may be employed. An ion flux may for instance be detected using well-established genetically encoded ion flux indicators such as a chemiluminescent ion sensor, aequorin and an engineered fluorescent protein. An ion concentration may also be measured directly using atomic absorption spectroscopy or labelled isotopes.

Where one or more membrane potential sensing dyes are used, fluorescence resonance energy transfer (FRET) can be exploited if a pair of dyes is used. A phospholipid-anchored first dye such as a coumarin and a second hydrophobic dye that rapidly redistributes in the membrane according to the transmembrane field may be used.

Where desired, a physiological ion that permeates TMEM16A in vivo may be replaced by a non-physiological ion that can permeate the pore defining a channel. As an illustration, other halogen ions such as bromide or iodide ions may be used, albeit TMEM16A has a lower affinity toward these ions. In this regard the selectivity for chloride is significantly higher at the lowest concentration of calcium.

To take account of conformational changes during gating it may be desired to use an assay format that can control channel gating in order to identify a compound with functional selectivity. In this regard techniques in automated electrophysiology, biochemical sensors, and plate readers provide a range of options for implementing an ion channel assay that can detect state-specific, or state-independent, channel modulation.

As an example, cells or oocytes expressing a polypeptide as disclosed herein in recombinant form can be contacted with a test compound, and the modulating effect(s) thereof can then be evaluated by comparing the TMEM16A-mediated response in the presence and absence of the test compound. The TMEM6A-mediated response of test cells, or control cells, typically cells that do not express the channel TMEM16A at all may also be compared to the presence of the compound.

In accordance with a particular embodiment of the method, a detectable electrophysiological response may be produced in a cell such as an oocyte. The method generally includes expressing a polypeptide as disclosed herein on a cell surface, such as an oocyte cell surface. The method may also include contacting the oocyte with one or more test compounds, and identifying the electrophysiological response.

In some embodiments an electrophysiological response can be detected using a technique called “patch clamping”. A small patch of cell membrane is generally isolated on the tip of a micropipette by pressing the tip against the membrane. It has been suggested that if a tight seal between the micropipette and the patch of membrane is established electric current may pass through the micropipette only via ion channels in the patch of membrane. If this is achieved the activity of the ion channels and their effect on membrane potential, resistance and current may be monitored. If the electric potential across the membrane remains constant, the current supplied to it is equal to the current flowing through ion channels in the membrane. The closing of ion channels in the membrane causes resistance of the membrane to increase. If the current applied remains constant; the increase of resistance is in direct proportion to an increase of electric potential across the membrane.

A method for identifying compounds that modulate TMEM16A activity (e.g., agonists and antagonists) typically requires comparison to a control. One type of a “control” cell or “control” culture is a cell or culture that is treated substantially the same way as the cell or culture exposed to the test compound. The only difference to the test cell or culture is that the control cell/culture is not exposed to a test compound. For example, in a method that is based on a voltage clamp electrophysiological technique, the same cell can be tested in the presence and absence of test compound, by merely changing the external solution bathing the cell. Another type of “control” cell or “control” culture may be a cell or a culture of cells that are identical to the transfected cells, except that the cells employed for the control culture do not express functional TMEM16A channels. In this situation, the response of test cell to test compound is compared to the response (or lack of response) of receptor-negative (control) cell to test compound, when cells or cultures of each type of cell are exposed to substantially the same reaction conditions in the presence of compound being analysed.

In some embodiments a measurement may be compared to a predetermined threshold value. A predetermined threshold value may in some embodiments be set on the basis of data collected from preceding measurements using compounds known to modulate, e.g. activate or inhibit TMEM16A activity. In some embodiments a certain percentile of such data may be used as a threshold value. The range of the values of a set of data obtained from cells can be divided into 100 equal parts, i.e. percentages of the range can be determined. A percentile represents the value within the respective range below which a certain percent of the data fall, in other words the percentage of the values that are smaller than that value. For example the 95th percentile is the value below which 95 percent of the data are found. In some embodiments TMEM6A activity may be regarded as decreased or low if it is below the 90^{th} percentile, or below the 80^{th} percentile. In some embodiments TMEM16A activity may be regarded as decreased or low if it is below the 70^{th} percentile.

In some embodiments a substrate for use in screening disclosed in European patent application EP 1 621 888 is used in connection with a biological membrane.

As explained above, a method of identifying a compound capable of modulating activity of a TMEM16A channel may involve using a non-human animal model for PH, including for PAH. The animal model may involve using a non-human animal, in particular a mammal, to which a compound known or suspected to modulate activity of a channel TMEM16A is being administered. Suitable animals to be used in a respective animal model are a dog and a rabbit. Further suitable animals to be used in a respective animal model are an ape and a monkey. Yet further suitable animals to be used in a respective animal model are a mouse and a rat. A Guinea pig is a further illustrative example of a suitable animal for such an animal model.

In case of an animal model, the non-human animal may have been deprived of adequate oxygen supply for a week or more. The non-human animal may for example have been exposed to a hypoxic ambience. Such an animal model has the pathology of PH and PAH, in particular hemodynamical characteristics such as an increase in RVSP and muscularization of pulmonary arteries as observed in PAH.

In case of an animal model, the animal may also be genetically engineered to develop PH, including PAH. In an animal model, an animal may also be genetically engineered in such a way that it shows the characteristics of PH, including PAH.

### Therapeutic Applications

An increased expression and activation of the Ca²⁺-activated Cl⁻ channel TMEM16A was found in the PASMC of IPAH patients. For the first time the clear therapeutic benefit of a chronic treatment with the TMEM16A inhibitor benzbromarone has been shown, as it caused reverse remodeling in two independent animal models of PH. These data also show that blocking or silencing of TMEM16A reversed the pathological membrane depolarization in vitro in the PASMC of IPAH patients, causes vasodilatation, and inhibits PASMC proliferation.

An increased TMEM16A expression was observed while screening for the compartment-specific regulation of Cl⁻ channels and transporters in the PA and primary cultured PASMC from IPAH patients. It has been demonstrated that these changes are consistent in human PASMC obtained from a large number of IPAH patients. In addition, for the first time the effects of TMEM16A inhibition and overexpression have been comprehensively evaluated. PASMC isolated from IPAH patients maintained their pathologic phenotype as they were depolarized and showed a TMEM16A upregulation similar to that found in the laser capture microdissected PA. Thus, the upregulation of TMEM16A belongs to the early events in the pathophysiologic mechanism of IPAH and is not a late secondary event. This concept explains a previous publication showing that endothelin-1 (ET-1), which plays an important role in PAH etiology, upregulates the TMEM6A protein in human PASMC (Hiram, R, et al., Am. J. Physiol. Heart Circ. Physiol. (2014) 307, H1547-1558). Increased TMEM16A expression lowers the proliferation of PAEC and augments the proliferation of PASMC while the apoptosis remains unaffected.

Moreover, in silico analysis by the inventors predicts binding sites for the transcription factor HIF1-α in the promoter region of TMEM16A. Exposure to hypoxia resulted in increased sarcolemmal TMEM6A protein levels in the PASMC of healthy donors and led to functional consequences by generating larger Ca²⁺ activated Cl⁻ current, compared to PASMC cultured under normoxic conditions. In addition, qRT-PCR analysis showed that the expression of the CFTR Cl⁻ channel gene was significantly lower in the pulmonary arteries of the IPAH patients. Since CFTR is also present in PASMC and is known to inhibit TMEM16A channel, one may speculate that the downregulation of CFTR may result in a further increase of TMEM16A channel function. Alternative splicing of the TMEM16A mRNA is another means to regulate the biophysical properties of TMEM16A channels: the presence of exons 6b, 13 and 15 are reported to influence the Ca²⁺ and Eₘ sensitivity as well as the speed of channel activation/deactivation (Ferrera, L, et al., J. Biol. Chem. (2009) 284, 33360-33368).

A TMEM16A inhibitor, which may for instance be a low molecular weight compound or an antibody may be provided in a composition which further includes a suitable carrier, excipient or diluent. In typical embodiments a respective composition includes an antibody described herein. Such a composition may, e.g., be a diagnostic, a cosmetic or a pharmaceutical composition. For therapeutic or cosmetic purposes, the composition is a pharmaceutical composition including a pharmaceutical carrier, excipient or diluent, i.e. not being toxic at the dosages and a concentration employed.

A TMEM16A inhibitor as described herein is useful as a medicament. Typically, such a medicament includes a therapeutically effective amount of a molecule as disclosed above. Accordingly, a respective molecule can be used for the production of a medicament useful in the treatment of PH. The respective PH may be PAH.

In one aspect, a method of treating PH is provided. The method may be a method of treating PAH. The method includes the steps of administering a pharmaceutically effective amount of a molecule as described herein, such as an antibody or a low molecular weight compound, to a subject in need thereof. In one embodiment, the pharmaceutical composition described above, which includes such pharmaceutically effective amount of the compound is administered to the subject. The medicament referred to above may be administered to a subject.

The subject in need of a treatment can be a human or a non-human animal. Typically the subject is a mammal, e.g., a mouse, a rat, rabbit, a hamster, a dog, a cat, a monkey, an ape, a goat, a sheep, a horse, a chicken, a guinea pig or a pig. In typical embodiments, the subject is diagnosed with a PH or may acquire such a disorder.

The TMEM16A inhibitor may be included in a pharmaceutical composition as indicated above. The pharmaceutical composition may be applied by one or more of various suitable routes of administration. Administration can for instance be conducted parenterally. In some embodiments administration is carried out intramuscularly. In some embodiments administration is carried out intravenously as a bolus or by continuous infusion. Administration is in some embodiments conducted intraarticularly. In some embodiments administration is done intrasynovially. Administration may in some embodiments be subcutaneously. In some embodiments administration is carried out topically, e.g., to the skin or the eye. Administration is in some embodiments carried out rectally. In some embodiments administration is done dermally such as intradermally, subcutaneously or transdermally. Administration can in some embodiments be performed locally. Further suitable modes of administration include, but are not limited to intracerebrally, intracerebrospinally, intrathecally, epidurally, or intraperitoneally; orally; urogenitally; intravitreally; systemically; intravenously; intraocularly; oticly; intranasally; by inhalation; sublingually; buccally, for example. Typical routes of administration are the topical, rectal, local, intranasal, intravenous and/or intradermal routes of administration.

### Article of Manufacture

In a further aspect, an article of manufacture such as a kit is provided. The article of manufacture includes matter, e.g. material, useful for (i) the treatment, prevention or delay of progression of PH, including PAH; (ii) diagnostic or (iii) cosmetic purposes. The article of manufacture may include instructions for use and one or more containers. Suitable containers include, for example, bottles, vials, syringes, cartridges, plates and test tubes and may be made from a variety of materials such as glass or plastic. At least one container holds a composition that includes a TMEM16A inhibitor as disclosed herein. The container may have a sterile access port. A respective container is typically labelled.

The reagents may for example be provided in predetermined amounts of dry powders, usually lyophilized, including excipients, which after dissolution will provide a reagent solution having the appropriate concentration. Other additives such as stabilizers and/or buffers may also be included. If the binding member is labelled with an enzyme, the kit will typically include the according substrates and cofactors.

The instructions for use may provide indications that the composition is used for the treatment, prevention and/or delay of progression of a disorder of choice; or instructions for performing a detection or diagnostic assay. The instructions may be provided on a label and/or on a package insert.

TMEM16A is a Ca²⁺-activated Cl⁻ channel implicated mainly in the growth and invasion of various cancers. Even though the channel is ubiquitously expressed throughout different tissues and organs, in the lung it is predominantly found in the epithelium and vasculature. The role of TMEM16A in the pulmonary circulation, particularly in the pulmonary arterial smooth muscle (PASMC) and endothelial cells (PAEC) has been largely unknown including its role in the pathogenesis of pulmonary arterial hypertension (PAH).TMEM6A has different effects on the cellular physiology of PAEC and PASMC. Upon overexpression, it depolarizes the resting membrane potential of both, PAEC and PASMC, demonstrating the downstream effects potentially being a consequence of the global depolarization. Furthermore, increased TMEM16A expression lowers the proliferation of PAEC and augments the proliferation of PASMC while the apoptosis remains unaffected. The tube formation is significantly lowered in TMEM16A-overexpressing PAEC. The findings demonstrate that increased TMEM16A expression is an important mechanism underlying vasoconstriction and remodelling of pulmonary arteries in PAH.

The listing or discussion of a previously published document in this specification should not necessarily be taken as an acknowledgement that the document is part of the state of the art or is common general knowledge.

While embodiments of the invention have been illustratively described, it is to be understood that the invention is not limited thereto but may be otherwise variously embodied and practiced within the scope of the appending claims. The invention may suitably be practiced in the absence of any element or elements, limitation or limitations, not specifically disclosed herein. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by exemplary embodiments and optional features, modification and variation of the invention embodied therein herein disclosed may be resorted to by those skilled in the art, and that such modifications and variations are considered to be within the scope of this invention.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein. In addition, where features or aspects of the invention are described in terms of Markush groups, those skilled in the art will recognize that the invention is also thereby described in terms of any individual member or subgroup of members of the Markush group.

The following are examples, illustrating the methods, uses and agents disclosed herein. It is understood that various other embodiments may be practiced, given the general description provided above.

### EXAMPLES

The examples illustrate techniques that can be used in a method and a use disclosed herein. The examples are presented in the form of an overview, with details of the methods and reagents following thereafter.

### Example 1—Upregulation of TMEM16A in the PASMC of IPAH Patients

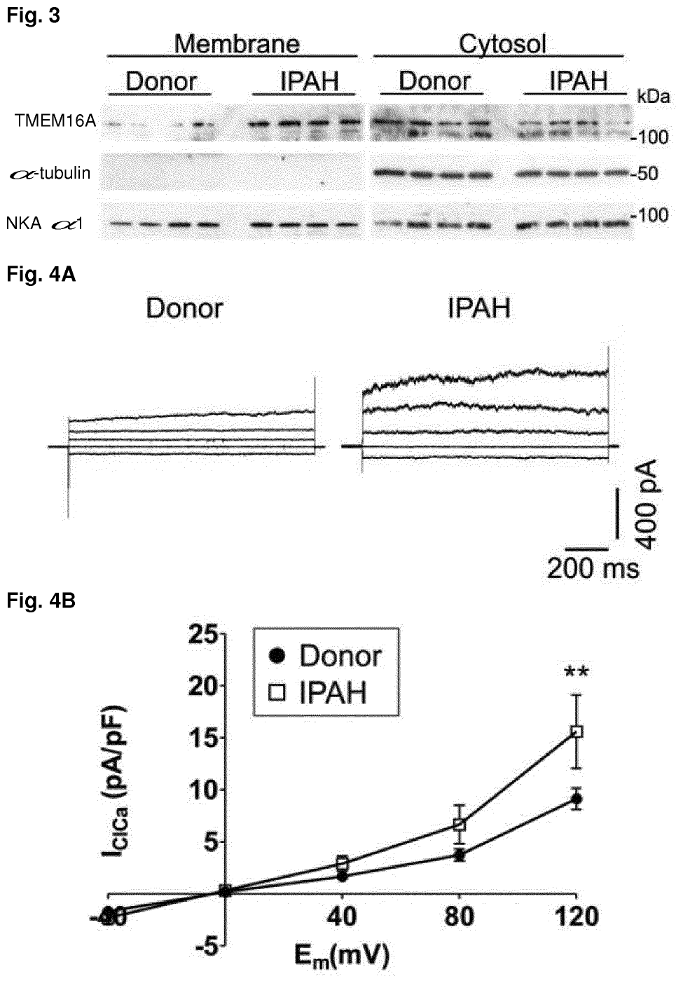

The present inventors tested the expression of Cl⁻ channel and transporter genes in the laser capture microdissected PA (LCM-PA) of healthy donors and patients suffering from IPAH. The mRNA for the Ca²⁺ activated Cl⁻ channel TMEM16A gene (ANO1) was upregulated in the LCM-PA as well as in primary PASMC isolated from IPAH patients (not shown). No significant regulation of the other channels/transporters was observed, except for the Cl⁻ channel CFTR, which showed lower expression in the PA of IPAH patients. Immunofluorescent stainings for α-smooth muscle actin (α-SMA) and TMEM16A on lung sections of healthy donors and IPAH patients (not shown) showed the presence of TMEM16A in the medial layer of human PA, both in donor lungs and in the remodeled arteries of IPAH lungs. Similarly, by immunofluorescent staining the expression of TMEM16A in the primary PASMC isolated from both donors and IPAH patients could be verified. To confirm the upregulation of TMEM16A on the protein level, the inventors performed western blots and detected a marked increase of TMEM16A in the membrane protein fraction of PASMC from IPAH patients (FIG. 3). In accordance with this, whole-cell voltage clamp measurements showed an increased Ca²⁺ activated Cl⁻ current (I_{ClCa}) in primary PASMC from IPAH patients, compared to the PASMC of healthy donors (FIG. 4A).

Next, upstream events that are likely to regulate TMEM6A were addressed. The recently reported novel zinc metalloprotease calcium-activated chloride channel activator 1 (CLCA1)-TMEM16A signaling remained intact in IPAH patients as the CLCA1 plasma level was not changed and the amount of CLCA1 in lung homogenates did not differ significantly in donor and IPAH lungs (not shown). Patients whose blood plasma was used for the CLCA1 concentration measurements were between 35 and 89 years old. Mean pulmonary arterial pressure of IPAH patients was between 39 and 51 Hgmm, with one exception. The exception was a patient where at the time of hemodynamic substudy Ca²⁺ channel antagonist therapy was stopped, however her mPAP values still remained below 25 mmHg. Mean pulmonary arterial pressure of IPAH patients was between 50 and 102 Hgmm with one exception. The exception was a patient for whom right heart catheterization data was not available, and the sPAP was estimated to be 154 Hgmm from echocardiography performed before lung transplant.

Treating the PASMC of healthy donors with CLCA1-containing conditioned medium did not affect the resting membrane potential (not shown). Furthermore, the expression of three exons reported to be subject of alternative splicing was quantified, since these may influence the biophysical properties of TMEM16A channels. No difference in the expression of splice variants was found between the PASMC of donors and IPAH patients (not shown). This agrees with the fact that no apparent difference in the biophysical properties of I_{ClCa} was observed in the voltage clamp recordings (FIG. 4B).

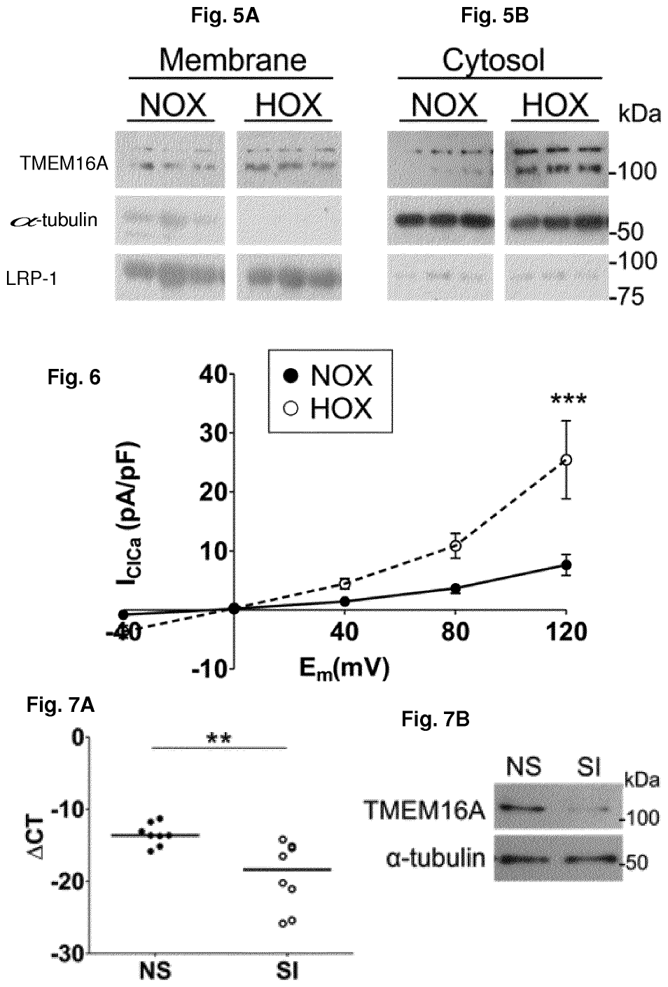

Based on initial in silico analyses data that predicted HIF-1α binding sites in the promoter region of the TMEM16A gene, factors activating HIF-1α were taken into consideration as upregulating TMEM16A. Therefore the effect of hypoxia on the TMEM16A protein expression/channel function was tested. 48 hours of hypoxia increased the amount of TMEM16A protein in the membrane fraction of the primary PASMC (FIG. 5a, 5b). These proteins formed functional channels, since a higher whole-cell I_{ClCa} was observed in cells exposed to hypoxia.

### Example 2—TMEM16A is Involved in the Chronic PASMC Membrane Depolarization in IPAH

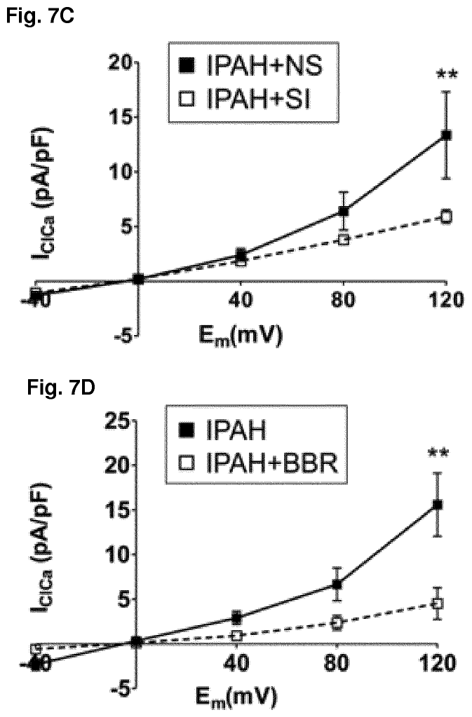

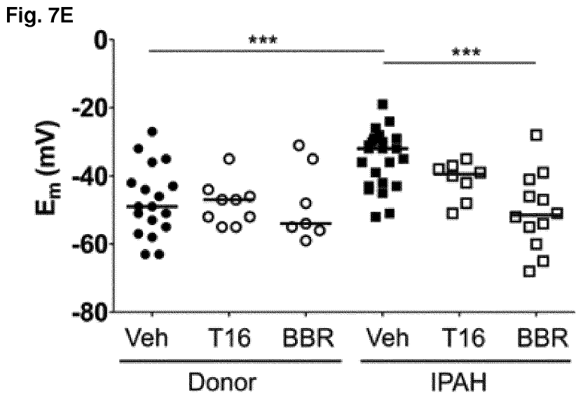

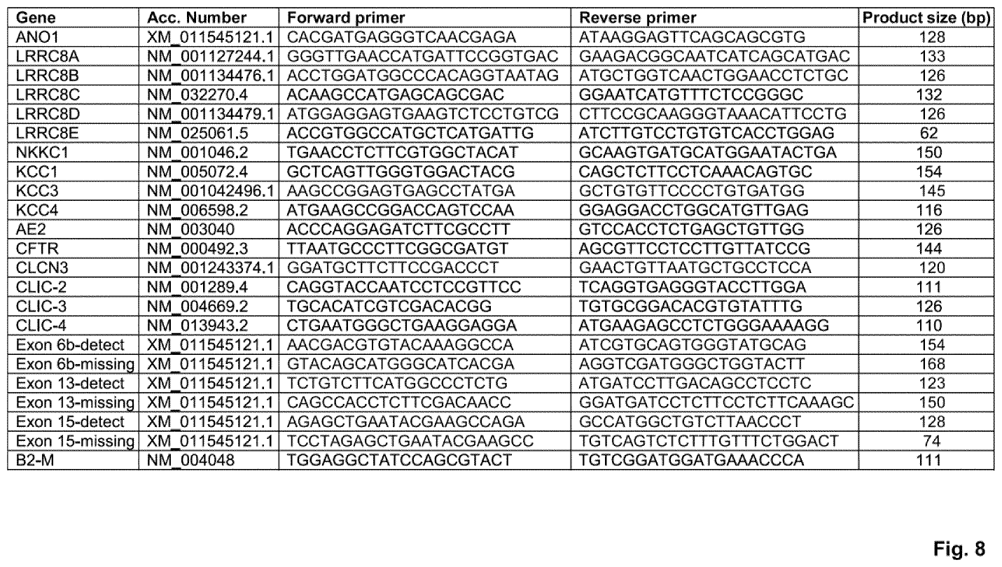

In order to determine the role of TMEM16A on membrane potential in human PASMC the inventors controlled the expression of TMEM16A and subsequently examined its impact in human PASMCs. Silencing of TMEM16A led to a decrease in TMEM16A mRNA, total protein and I_{ClCa} in primary PASMC compared to PASMC treated with non-silencing control RNA (FIGS. 7A, B and C). Similarly, benzbromarone (BBR), a recently identified potent inhibitor of TMEM16A channels, significantly decreased whole-cell I_{ClCa} measured in the primary PASMC of both donors and IPAH patients (FIG. 7D). The resting membrane potential (Eₘ) of the primary PASMC isolated from IPAH patients was significantly more depolarized than the Eₘ of the donor PASMC (FIGS. 7E and F). Benzbromarone as well as another structurally non-related selective TMEM16A blocker, T16Ainh-A01 (abbreviated as T16 in FIG. 7E) reversed the Eₘ of IPAH-PASMC close (T16Ainh-A01) or completely (BBR) to the levels of PASMC isolated from healthy donors, while these substances had no effect on the PASMC of donors (FIG. 7E). Silencing of TMEM16A in IPAH-PASMC rescued the Eₘ of the IPAH-PASMC without significantly affecting donor PASMC (FIG. 7F). As a second approach, the inventors overexpressed TMEM16A in human donor PASMC.

### Example 3—Acute Vasorelaxant Effect of the TMEM16A Inhibitor Benzbromarone

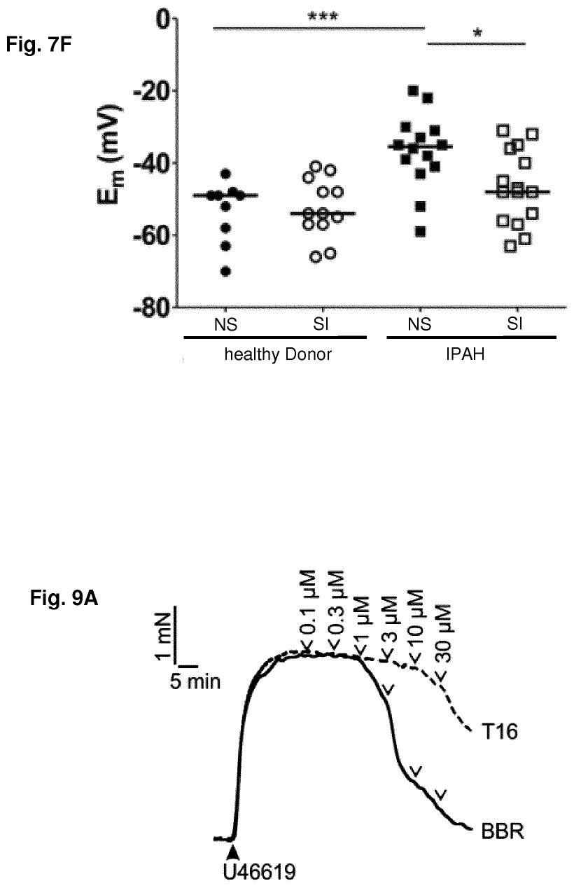

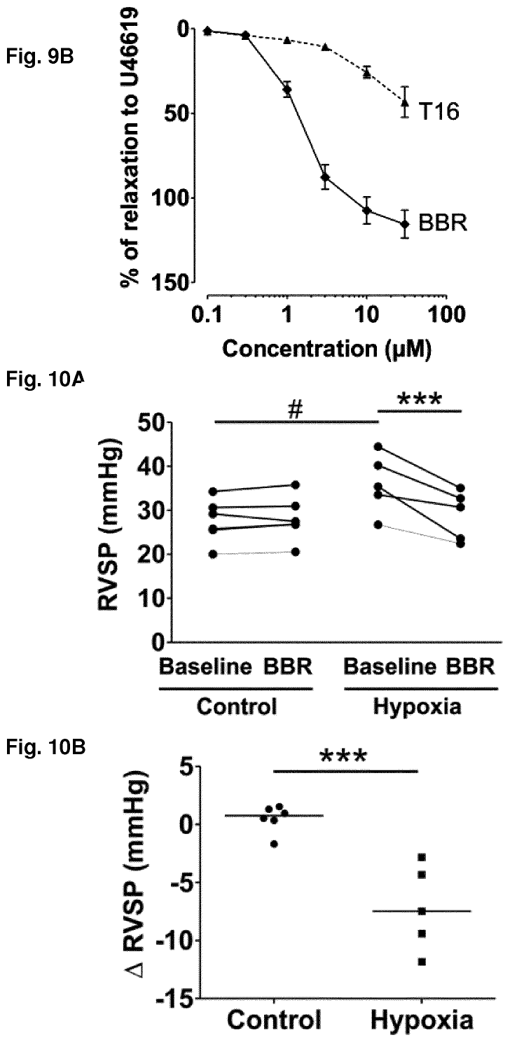

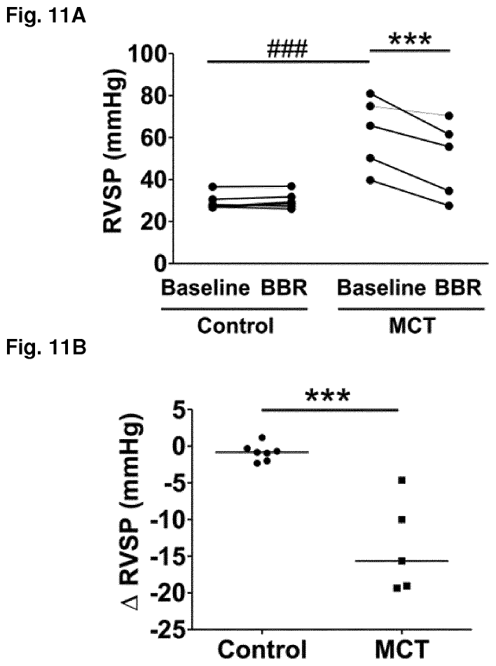

The finding that TMEM16A inhibition reverses membrane depolarization in the PASMC of IPAH patients prompted the inventors to evaluate the effect of TMEM16A inhibition on the pulmonary circulation in both animal models and humans. In order to determine the effective dose for acute vasorelaxation, the inventors examined the TMEM16A inhibitor mediated PA vasodilator response ex vivo. Both T16Ainh-A01 and benzbromarone caused a dose-dependent vasorelaxation of U-46619 preconstricted isolated mouse PA (FIGS. 9A and B). In the second approach, the more potent and better soluble benzbromarone was applied in vivo. Under continuous in vivo hemodynamic monitoring, benzbromarone was applied as an intravenous bolus in two different animal models of PH: in hypoxia-exposed mice (FIGS. 10A and B) and in monocrotaline (MCT)-treated rats (FIGS. 11A and B) to measure its acute effects. Benzbromarone, at a concentration that effectively dilated PAs ex vivo, caused a significant decrease in the RVSP in both models without affecting RVSP in the control animals. To assess the acute pulmonary vasodilatative potency of benzbromarone in humans, 10 patients with severe IPAH were enrolled and 200 mg benzbromarone were administered orally as a single dose during a routine right heart catheter investigation. 200 mg is the maximum approved single oral dose for the treatment of gout in humans. Patient demographic and hemodynamic data are described in Supplementary Table 4. There was a slight increase in mPAP and PVR after 120 min as expected after application of a placebo with an indwelling right heart catheter but no further changes in the pulmonary or systemic hemodynamics occurred (Supplementary Table 5). No clinical adverse effects were observed throughout the study.

### Example 4—Chronic Benzbromarone Treatment Reverses the Development of PH in Animal Models

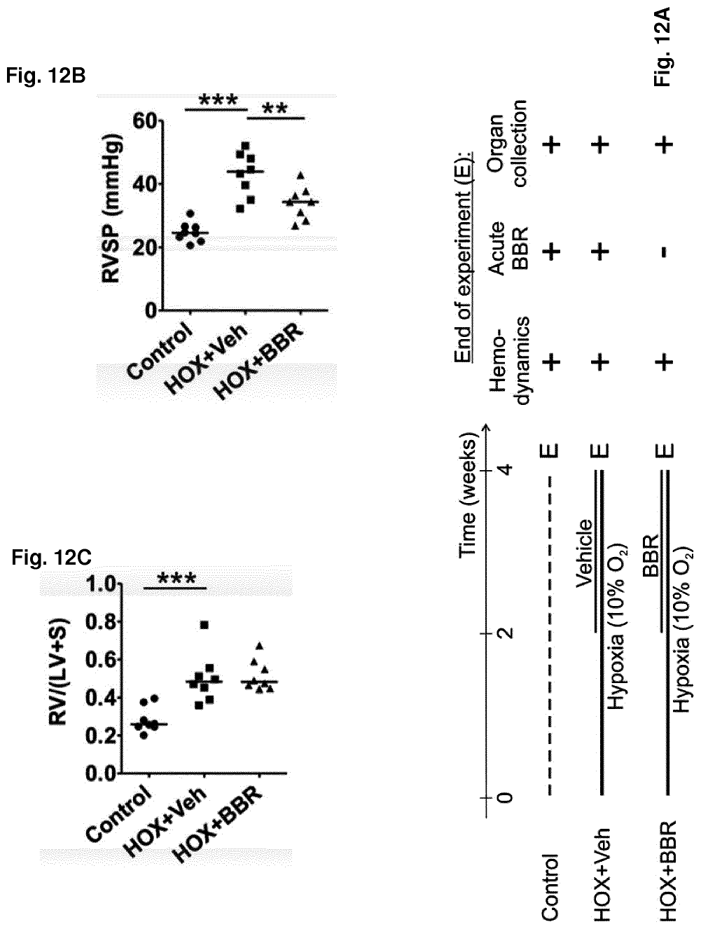

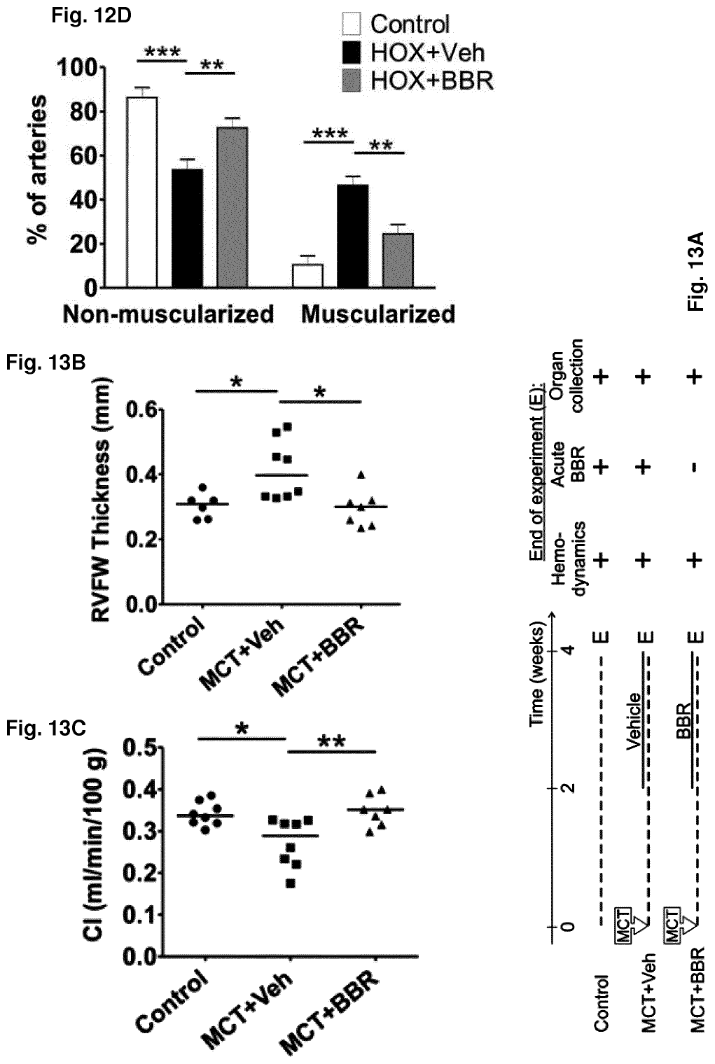

To assess the therapeutic potency of benzbromarone for reverse remodeling, benzbromarone and vehicle (Veh) were applied as subcutaneous slow-release pellets in two different animal models of PH at a dose corresponding to doses used for treatment in models of hyperuricemia in rodents or monkeys. A schematic diagram of the experiments using hypoxia-exposed mice is given in FIG. 12A, and is further explained below. After 4 weeks of hypoxia, significant increases in the RVSP and in the Fulton index were observed in placebo-treated mice (FIGS. 12B and C). Both were lowered by the long-term benzbromarone treatment, without altering the systemic arterial pressure (SAP). With respect to vascular remodeling, benzbromarone treatment reversed the hypoxia-induced muscularization of pulmonary arteries, as shown by a reduced ratio of fully vs non-muscularised arteries when compared to the HOX+Veh group (FIG. 12D).

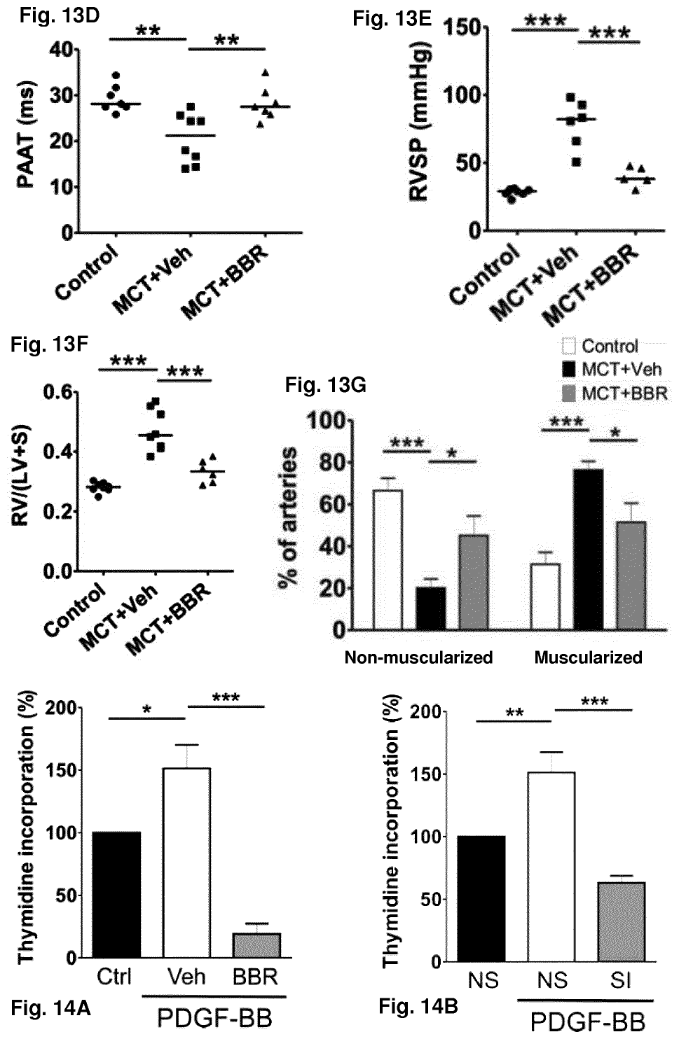

Similar to the mouse study, rats were randomized into a control group and two MCT-treated groups (FIG. 13A). The MCT-induced increase in right ventricular free wall thickness (RVFW Thickness) and the decrease in pulmonary artery acceleration time (PAAT) and cardiac index (CI) were completely reversed by the chronic benzbromarone treatment (FIG. 13B to D). Compared to the MCT+Veh group, RVSP and RV hypertrophy were significantly decreased in the benzbromarone treated rats (FIGS. 13E and F) without changes in SAP. The markedly reduced number of fully muscularized arteries and the increased number of non-muscularized arteries indicated a potent and nearly complete reverse-remodeling induced by benzbromarone (FIG. 13G).

### Example 5—Inhibition of TMEM16A Results in Reduced PASMC Proliferation

Immunohistological stainings of mouse and rat lung sections for the proliferation marker PCNA showed an increased number of PCNA positive nuclei in the medial layer of the PA in both hypoxic mice and MCT treated rats, compared to the normoxic/vehicle-treated controls. In parallel with the hemodynamic improvement, a reduced number of PCNA positive nuclei was observed in the BBR-treated animals (not shown). Finally the loss of TMEM6A function and of TMEM6A expression reverses human PASMC proliferation. Both treatment with BBR and siRNA against TMEM16A led to a decrease in PDGF-BB induced PASMC proliferation (FIGS. 14A and B).

### Methods

Methods included Cas3/7 activity apoptosis assay, Matrigel tube-formation assay, Thymidine incorporation proliferation assay, DiBAC4(3) resting membrane potential determination assay.

### Human Lung Samples

Human lung tissue samples were obtained from patients with idiopathic pulmonary arterial hypertension (IPAH) who underwent lung transplantation at the Department of Surgery, Division of Thoracic Surgery, Medical University of Vienna, Vienna, Austria. The protocol and tissue usage were approved by the institutional ethics committee (976/2010) and written patient consent was obtained before lung transplantation. The patient characteristics included: age at the time of the transplantation, weight, height, sex, mean pulmonary arterial pressure (mPAP) measured by right heart catheterization, pulmonary function tests, as well as the medical therapy. The chest computed tomography (CT) scans and right heart catheterization (RHC) data were reviewed by experienced pathologists and pneumonologists to verify the diagnoses. Healthy donor lung tissue was obtained from the same source.

### Laser Capture Microdissection of Pulmonary Arteries

Laser capture microdissection (LCM) of 17 donor lungs and 14 lungs from IPAH patients, as well as mRNA subtraction and cDNA preparation were performed as previously described in the literature. The intima and media layers of pulmonary arteries of 100-500 m diameter were collected.

### Primary Human Pulmonary Arterial Smooth Muscle Cell (PASMC) Isolation

The isolation and culture of PASMC has been performed according to Stulnig et al (Stulnig, G, et al., Atherosclerosis (2013) 230, 406-413). VascuLife Complete SMC Medium (LifeLine Cell Technology, Frederick Md., USA) containing 10% FCS (Biowest, Nuaillé, France) and 0.2% antibiotics/antimycotics (LifeLine Technology) was used for culturing. Immunofluorescent labelings (described in details below) were performed routinely on these cells to test for α-smooth muscle actin positivity and the lack of signal for von Willebrand factor. For all experiments, PASMC underwent no more than one freeze/thaw cycle (freezing medium: VascuLife Complete SMC Medium containing 15% FCS and 10% DMSO) and cells were used no longer than the 5 passage. Donors from which PASMC were isolated included both male and female subjects. IPAH patients from which PASMC were isolated included both male and female subjects. mPAP of the IPAH patients ranged between 50 and 102 Hgmm. For one patient who had undergone lung transplantation, no right heart catheterization data was available. sPAP was estimated to be 154 Hgmm from echocardiography performed before lung transplant.

### Cloning of pQXCIP-TMEM16A_GFP

### PCR for TMEM6A_GFP Amplification

cDNA used was from a human podocyte library, TMEM16A was equipped with an N-terminal GFP, and the respective construct was inserted into the retroviral expression vector PQCXIP (Clontech Laboratories, Takara Bio Europe, Saint-Germain-en-Laye, France, Cat. No. 631516).

The template is pAno1-GFP. Primers used were:

GCGGCCGCCACCAGGCGCGCCATGAGGGTCAACGAGAAGTAC (ANO1sh
fw)
and
TTAATTAATTACTTGTACAGCTCGTCCAT (ANO1sh revmgfp)

The polymerase used was Accu Prime pfx polymerase (with proofreading function)
The temperature pogramme was run as follows: D 94° C. 5′
D94° C. 30″
A 55° C. 30″
E 68° C. 4′
E 68° C. 7′

The PCR product was purified using a 1% agarose gel by means of the kit Zymoclean™ Gel DNA Recovery Kit (Zymo Research, Freiburg, Germany).

### Preparation of Ligation with the Vector pENTR-TOPO, and Transformation

The template is pENTR-TOPO hAno1 GFP. Restriction enzymes used on the vector were PacI and NotI. The obtained vector was purified using a 1% agarose gel. The PCR product was purified on a column using the kit DNA Clean & Concentrator™ (Zymo Research, Freiburg, Germany). Ligation of vector (pENTR-TOPO) and insert (TMEM16A_mGFP) was done using T4 ligase (Fermentas GmbH, St. Leon-Rot, Germany) at a ratio of 1:1 (vector:insert). E. coli DH10B was transformed using 5 μl of the ligation product.

### Screening of the Clones

The obtained vector was exposed to minipreparation using the kit Zyppi™ Plasmid Miniprep Kit (Zymo Research, Freiburg, Germany). Digest using restriction enzymes PacI und NotI was done on samples to identify suitable clones. Two clones were picked, and underwent midipreparation und sequencing of the plasmid.

```
Seq-Primer used:
  1 - GGACCCTGGTCAGGAGGGTGC,
  2 - CGGGTTTGTGAAAATCCATGC,
  4rev - GGCCAATGGTGTGTACGCGGC,
  5 - GAGACACAATATCACCATGTGC,
  6 - AAGAGACTGACAAAGTGAAGC,
  7 - CTCCTGGACGAGGTGTATGGC,
  8 - ATCCAGCTCAGCATCATCATGC,
10 - TGGACCTGGGCTACGAGGTGC,
11 - AGACAGCCTCGGCAGCCC AGC,
12 - GCCGAGGTGAAGTTCGAGGGC.
```

The identified sequences matched the sequence of Genbank accession number A1B845669.1, as of 5 Feb. 2014, to 100%.

### Preparation of Cloning into the Vector pQXCIP

Vector pQXCIP and pENTR-TOPO_TMEM16A_GFP were exposed to restriction enzymes PacI und NotI. In addition, the vector was exposed to Antarctic Phosphatase (New England Biolabs, Ipswich, Mass., U.S.). The vector and the insert were purified by means of an agarose gel (1%) and the Zymoclean™ Gel DNA Recovery Kit. Vector (pQXCIP) and insert (TMEM16A_mGFP) were ligated using T4 ligase (Fermentas GmbH, St. Leon-Rot, Germany) at a ratio of 1:1. E. coli DH10B was transformed using 5 μl of the ligation product.

### Screening of the Clones

The resulting vector underwent minipreparation using the kit Zyppi™ Plasmid Miniprep Kit (Zymo Research, Freiburg, Germany). Digest using restriction enzymes PacI und NotI was done and sequencing of the clones picked was done as described above.

### qRT-PCR

### Laser Capture Microdissected Human Pulmonary Arteries

The expression of ion channels and transporters was analyzed with qRT-PCR using the QuantiFast SYBR PCR reagent (Qiagen, Hilden, Germany). Primer pairs (Eurofins, Graz, Austria) were designed to span at least one exon-exon boundary to avoid the amplification of genomic DNA. The specificity of all primers as well as the length of the amplicon were confirmed by melting curve analysis and by running the products on 2% agarose gels, respectively. Expression levels (CT values) of the target genes were normalized to 2-microglobulin expression as a reference gene.

### Primary Human Pulmonary Arterial Smooth Muscle Cells

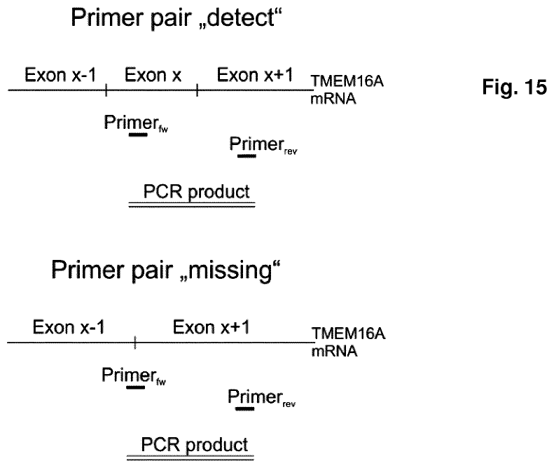

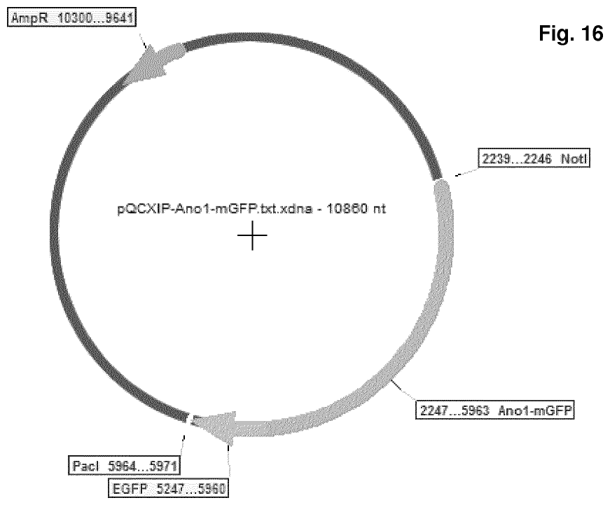

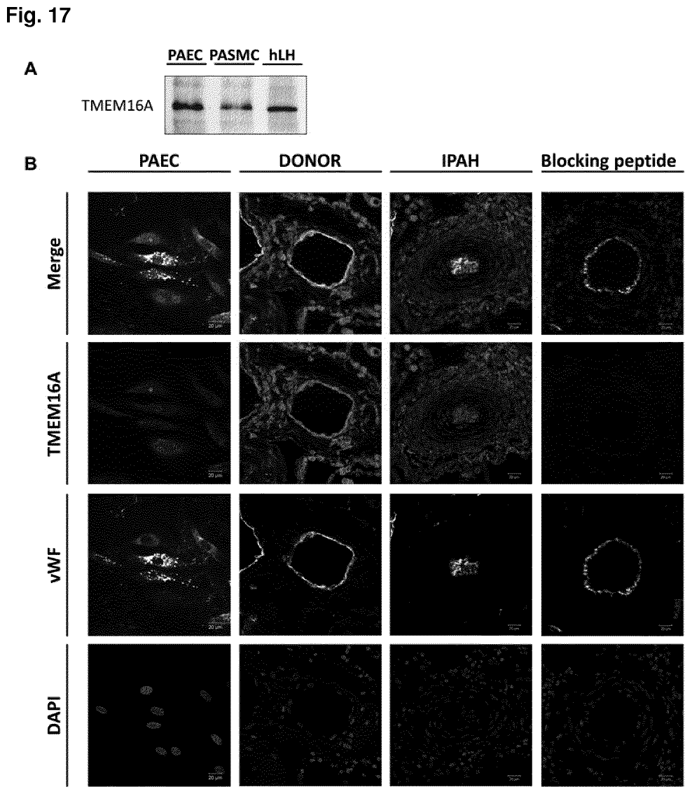

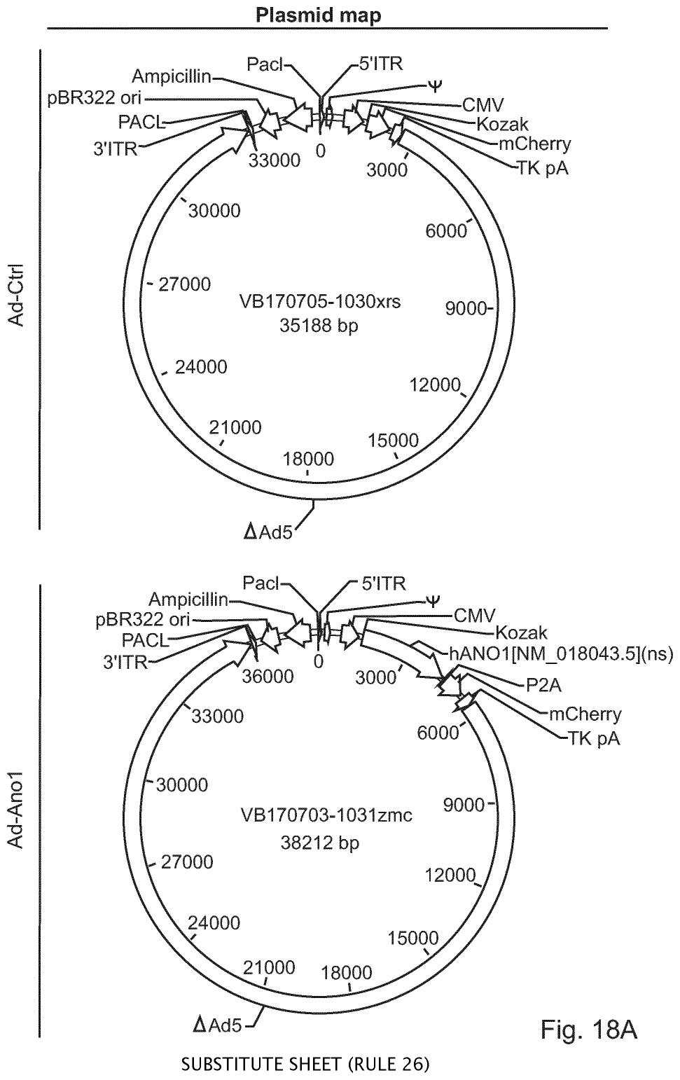

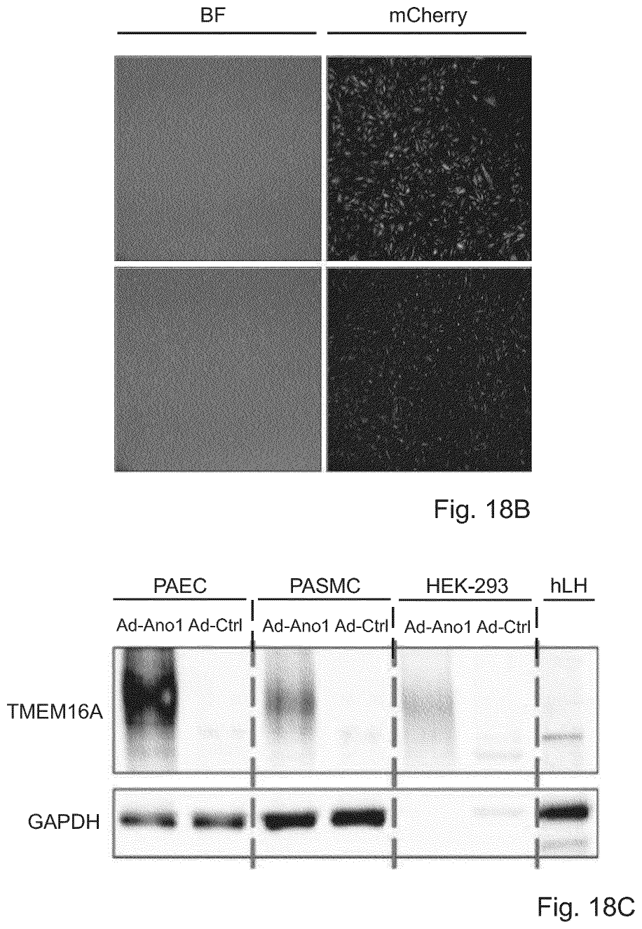

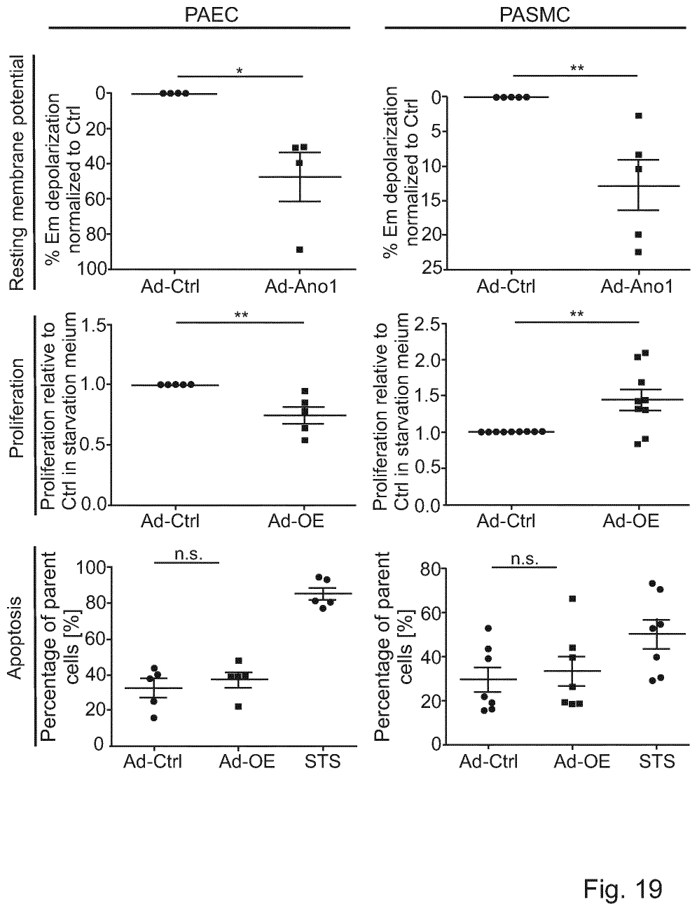

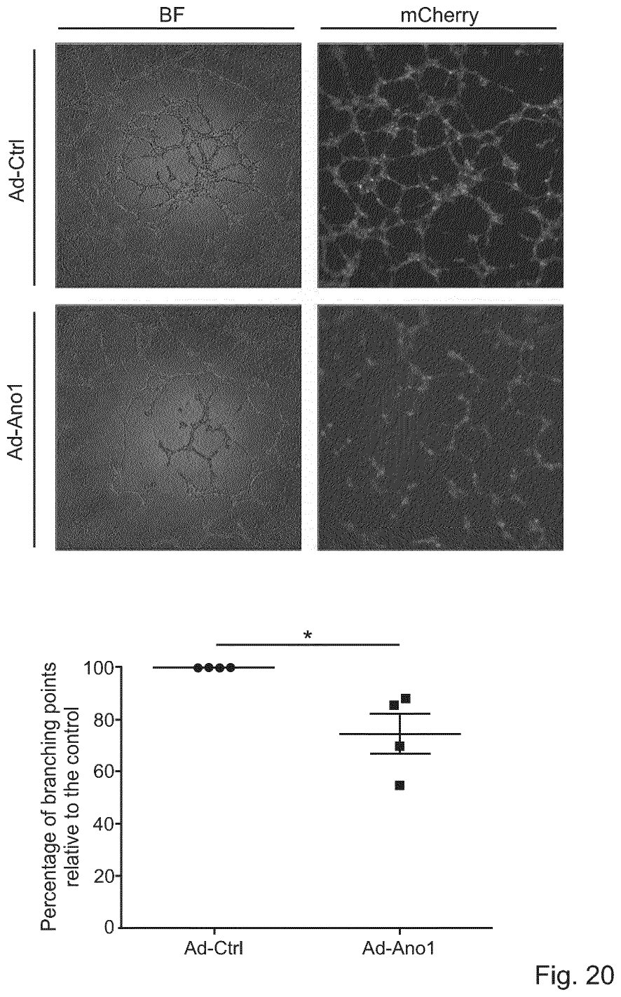

PASMC were grown until confluence, serum-starved overnight using VascuLife Basal Medium containing 0.2% antibiotics, then RNA was isolated using the PeqGOLD Total RNA kit (PeqLab, Erlangen, Germany) and transcribed into cDNA with the iScript reagent mix (Bio-Rad, Hercules Calif., USA). qRT-PCR was performed as described above. To assess ANO1 expression, the primers targeted the boundary of exons 1 and 2. As this region is not reported to be subject of alternative splicing, these primers amplified all splice variants. In order to study alternative splicing, primer design principles are depicted in FIG. 15. Exon detecting (“detect”) primers were designed so that one of the primers should attach to the studied exon. Primers detecting the absence of an exon (“missing”) were created so that one of the primers should bind to the corresponding exon-exon boundary only when the studied exon is missing.

### Immunofluorescence Staining for TMEM16A

Formalin-fixed paraffin-embedded human lung tissue blocks were cut to 3.5 μm thick sections and antigen retrieval was performed with Dako Target Retrieval Solution pH 9.0 (Dako, Glostrup, Denmark) at 95° C. After blocking with 3% BSA, sections were immunolabeled for α-smooth muscle actin (EB06450, Everest Biotech, Upper Heyford, UK) and TMEM6A (#ACL-011, Alomone Labs, Jerusalem, Israel) overnight, followed by a labeling with Alexa Fluor 488 conjugated anti-rabbit and Alexa Fluor 555 conjugated anti-goat secondary antibodies (Life Technologies, Carlsbad Calif., USA). Slides were covered using Vectashield mounting medium with DAPI (Vector Laboratories, Peterborough, UK) and imaged with a Zeiss LSM 510 META laser scanning confocal microscope. Duplicates were processed without the primary antibody and/or with the primary antibody pre-incubated with the immunogen peptide as negative controls.

PASMC seeded on chamber slides were fixed with formalin, permeabilized with 0.1% Triton X-100 and then processed the same way as reported for the formalin-fixed lung tissue.

### Analysis of Protein Expression

### Human PASMC Sarcolemmal Proteins

Human PASMC grown on 10 cm Petri dishes were labeled with 1 mg/ml EZ-link NHS-SS-biotin (Thermo Scientific, Waltham Ma, USA) for 1 h at 4° C. and rinsed 3 times with PBS (137 mM NaCl, 2.7 mM KCl, 10 mM Na₂HPO₄, 2 mM KH₂PO₄, pH 7.4) containing 100 mM glycine. Cells were then solubilized in cell lysis buffer (50 mM Tris pH 7.4, 100 mM NaCl, 50 mM NaF, 5 mM-glycerophosphate, 2 mM EDTA, 2 mM EGTA, 1 mM sodium orthovanadate, 0.1% Triton X-100) containing protease inhibitor cocktail (Roche Diagnostics, Basel, Switzerland). Protein concentration was determined using a Pierce BCA Protein Assay Kit (Thermo Scientific). 100 μg of protein per sample were incubated overnight at 4° C. with end-over-end shaking in the presence of Neutravidin Agarose Resin Beads (Thermo Scientific). After centrifugation, the supernatant was carefully removed from the beads and frozen as the cytosolic fraction. In order to purify the membrane protein fraction, beads were washed and resuspended in 25 μl of 2× Laemmli sample buffer (10% SDS, 20% Glycerol, 0.2 M Tris-HCl, 0.05% bromophenol blue, 10% beta mercaptoethanol). Biotinylated cell surface proteins as well as the cytosolic fractions were separated on a 10% SDS-PAGE, followed by electrotransfer to a PVDF membrane (GE Healthcare, Little Chalfont, UK). After blocking the membrane with 5% non-fat milk in TBS-T (5 mM Tris-Cl, 150 mM NaCl, 0.1% Tween 20, pH 7.5), the membrane was labeled for TMEM16A (ab53212, Abcam, Cambridge, UK), followed by incubation with horseradish peroxidase conjugated secondary antibody (Dako, Glostrup, Denmark). Final detection of the proteins was performed using an ECL Plus Kit (GE Healthcare, Little Chalfont, UK) and a ChemiDoc™ Touch Imaging System. To verify the purity of the membrane fraction and to determine the amounts of protein loaded on the gel, blots were stripped with Restore™ Plus Western Blot Stripping Buffer (Thermo Scientific) and reprobed using antibodies against NA/K ATPase (ab 74945), LRP-1 (ab92544, both from Abcam) or α-tubulin (11H10 Cell Signaling Technology—New England Biolabs, Hitchin, UK).

### Human PASMC Total Protein

Human PASMC were grown on 6-well plates until confluence, serum-starved overnight, and harvested using RIPA buffer (Sigma) supplemented with protease inhibitors (Roche). The cell lysate was sonicated and centrifuged for 10 min at 5000 g. Protein concentration of the supernatant was determined using the Pierce BCA Protein Assay Kit (Thermo Scientific), then 20 μg protein from each sample was mixed with 10× Laemmli sample buffer and ran on 10% SDS-PAGE. Transfer, immunolabeling and signal detection were carried out as described above.

### Manipulation of TMEM16A Expression

For TMEM16A silencing, human PASMC were serum-starved for 3 hours, then transfected either with 100 nM siRNA against TMEM16A (SMART Pool: ON-TARGET plus ANO1 siRNA, Cat #L-027200-00-0005, Dharmacon, Lafayette Co, USA) or with non-silencing control RNA (ON-TARGET plus Non-targeting pool, Cat #D-001810-10-05, Dharmacon) using the Effectene transfection reagent kit (Qiagen) in serum-free medium. After 6 hours, the reagent mixture was replaced with Complete SMC Medium. We observed a significant reduction of the TMEM16A mRNA and protein 48 and 72 hours post-transfection, respectively (FIGS. 7A and B). Similarly, 72 hours post-transfection a significant decrease in I_{ClCa} was observed (FIG. 7C).

The vector used for TMEM6A overexpression was constructed at the University of Miinster, by cloning the TMEM16A gene from a human podocyte cDNA library into a pQCXIP expression plasmid. Transfection was performed in Complete SMC Medium overnight, following the instructions of the Effectene transfection reagent kit (Qiagen), then the reagent mixture was changed to Complete SMC Medium. As a control, cells from the same batch were transfected with the empty pQCXIP plasmid. A significant elevation in the TMEM16A mRNA was observed 48 hours post-transfection, accompanied by an increased TMEM16A protein level and I_{ClCa} density 72 hours post-transfection.

### Electrophysiology

### Membrane Potential (Eₘ) Measurement

PASMC were grown on glass coverslips and serum starved for 24-48 hours. The coverslips were perfused with a bath solution composed of (in mM) NaCl 141, KCl 4.7, HEPES 10, glucose 10, CaCl₂ 1.8, MgCl₂ 1.2, pH 7.4 (NaOH) at room temperature. Micropipettes were pulled of borosilicate glass capillaries (GC150F-10, Harvard Apparatus Ltd, Edenbridge, Kent, UK) with a P-2000 electrode puller (Sutter Instruments, Novato Ca, USA), and fire polished using a micro-forge (MF-830, Narishige, Tokyo, Japan) to give a final resistance of 3-5 MΩ when filled with the pipette solution (composition in mM: NaCl 10, KCl 125, K₂ATP 5, HEPES 10, EGTA 5, MgCl₂ 4, pH set to 7.2 with KOH). Eₘ was measured with a HEKA EPC10 patch clamp amplifier (Dr. Schulze GmbH, Lambrecht, Germany) in the current clamp mode as described by others (Mason, M J, et al., Biophys. J. (2005) 88, 739-750; Perkins, KL, J. Neurosci. Methods (2006) 154, 1-18).

The liquid junction potential was calculated with the Clampex 10.0 software (Molecular Devices, Sunnyvale Calif., USA) and an online correction was made for each recording. Both at baseline and during perfusion with 30 μM benzbromarone (BBR, Sigma, St Louis Mo., USA) or 10 μM T16Ainh-A01 (Tocris, Bristol, UK), only those recordings in which (1) the Eₘ was constant for at least 3 minutes, and (2) the seal resistance was constantly high (>5 GΩ) were used for the analyses.

### Measurement of Whole-Cell Ca²⁺-Activated Cl Current (I_{ClCa})

For the measurement of whole-cell I_{ClCa}, PASMC grown in T-25 tissue culture flasks were freshly harvested with StemPro Accutase (Life Technologies), centrifuged (300 g, 5 min) and resuspended in Complete SMC Medium. PASMC were stored at 4° C. until use and were allowed to attach to glass coverslips for 15-30 min at room temperature before the measurements. Cells were perfused with a bath solution composed of (in mM) NaCl 150, glucose 10, HEPES 10, CaCl₂ 1, MgCl₂ 1, pH 7.4 (NaOH). The pipette solution contained (in mM) CsCl 110, TEA-Cl 20, EGTA 5, HEPES 10, Na₂ATP 1, CaCl₂ 4.68, MgCl₂ 1, pH 7.2 (NaOH). The free Ca²⁺ concentration of this solution was 2 μM as calculated with the MaxChelator software. In order to minimize K⁺ current contamination of the recordings, after the formation of whole-cell configuration the bath solution was switched to a bath composed of (in mM) NaCl 140, glucose 10, HEPES 10, TEA-Cl 10, CaCl₂ 1, MgCl₂ 1, pH 7.4 (NaOH). In order to measure I_{ClCa}, the command potential was stepped from a 0 mV holding potential to −40, 0, +40, +80 and +120 mV for 1.5 s, allowing 0.5 s recovery time at the holding potential between each step. The average current measured between 750 and 1500 ms of each voltage step was plotted against the holding potential. Due to the almost symmetrical Cl-concentration of the bath and pipette solutions, the reversal potential (Eᵣₑᵥ) for Cl⁻ was expected to be around zero. Current recordings showing an Eᵣₑᵥ significantly different from zero were considered as contaminated with K⁺ currents and thus discarded. To study the effects of hypoxia, PASMC were incubated for 48 hr in a hypoxic incubator (1% O₂, 5% CO₂, 37° C.), and harvested with StemPro Accutase as described previously, except that all solutions were pre-incubated in a hypoxic incubator for 1 hour, and harvesting was performed in a hypoxic workstation (1% O₂, 5% CO₂). The suspended PASMC were aliquoted into screw-cap glass tubes and kept at 4° C. until use. Before use, cells were allowed to attach to glass coverslips for 15-30 min in the hypoxic workstation at room temperature. Bath solutions were continuously bubbled with N₂. Cells from the same batch that were cultured, harvested and recorded under normoxic conditions served as controls.

### Assessment of PASMC Proliferation In Vitro

Human PASMC were seeded on 96-well plates. Cells were serum-starved for 12 hours, then underwent a 30 min pre-treatment with benzbromarone (30 μM) or vehicle. After the pre-treatment, serum-free SMC Medium containing PDGF-BB (10 ng/ml, Sigma) and benzbromarone was applied. The proliferation was determined by [³H]-thymidine incorporation (BIOTREND Chemikalien GmbH, Cologne, Germany) as an index of DNA synthesis, and detected with a scintillation counter (Wallac 1450 MicroBeta TriLux Liquid Scintillation Counter & Luminometer, PerkinElmer, Waltham Mass., USA). Each independent experiment was performed in five replicates using PASMC isolated from six different donor lungs.

In experiments using TMEM16A silencing, cells were first seeded on 6-well plates and transfected with TMEM16A siRNA or control non-silencing RNA as described above. Cells were re-seeded into 96-well plates 24 hours after transfection, starved for 12 hours, then the induction and measurement of proliferation was performed as detailed above.

### Treatment of Human PASMC with CLCA1-Containing Conditioned Medium

The production and use of human CLCA1 protein containing conditioned medium was performed as described by a previous study (Sala-Rabanal, M, et al., Elife (2015) 4, 10.7554/eLife.05875), using HEK-293 cells to produce the conditioned medium. According to this study as well as our previous experience, HEK-293 can be transfected with high efficiency and have negligible endogenous CLCA1 expression. The identity of HEK-293 cells (#300192, CLS Cell Lines Service GmbH, Eppelheim, Germany) was verified by STR analysis and samples were negative for Mycoplasma. Cells were grown until 50% confluency in DMEM F-12 complete medium (ThermoFischer Scientific, Vienna, Austria) containing 10% FCS (Lactan, Graz, Austria), 1% L-Glutamine and 0.2% antibiotics. The plasmid used for transfection was a kind gift of Ute Schuessler (Bayer) and was constructed by cloning the human CLCA1 gene into a pcDNA 3.1(+) vector.

The HEK-293 cells were transfected overnight either with the CLCA1 gene containing or with the empty plasmid, using the Effectene transfection reagent kit (Qiagen) and DMEM F-12 complete medium. On the next day, the medium was changed to serum-free SMC Medium. The resulting conditioned media were collected 24 hours later and applied on the PASMC of healthy donors. After 24 hours of incubation, the Em of the PASMC was measured as described above. To confirm the presence of CLCA1 in the conditioned medium, 20 μL of conditioned medium as well as control medium (collected from cells transfected with the empty plasmide) were analyzed with western blot as described above, using a CLCA1 antibody (ab108851, Abcam).

To verify equal sample loading, the membrane was stained with Coomassie Blue. A band at approx. 66 kDa appeared in both samples. We assumed this band corresponded to albumin and showed an equal density for both media as a verification for equal protein loading.

### Measurement of Pulmonary Artery Tone Ex Vivo

Pulmonary artery rings were isolated from male C57BL/6J mice (20-25 weeks old, Charles River, Wilmington Mass., USA), as described by Jain et al. (Jain, P P, et al., Int. J. Nanomedicine (2014) 9, 3249-3261). Following stable contraction (approx. 30 minutes post-addition of U-46619, 30 nM), the arteries were subjected to cumulatively increasing concentrations (0.1 to 30 μM) of the TMEM6A blockers T16Ainh-A01 (Tocris) and benzbromarone (Sigma) in order to analyze the concentration-response relationships.

### Animal Models of Pulmonary Hypertension (PH)

All animal studies conformed with the EU guidelines 2010/63/EU and were approved by the University Animal Care Committee and the federal authorities for animal research approved the study protocol (approval numbers: BMWFW-66.010/0144-WF/V/3b/2014, BMWFW-66.010/0076-WF/V/3b/2015). The ARRIVE guidelines were considered and all measures were taken to keep animal suffering to a minimum. Based on the 3R principle, mice of the control and HOX+Veh groups as well as rats from the control and MCT+Veh groups were utilized to assess the acute effects of benzbromarone (described below and illustrated on FIGS. 12A and 13A), in order to reduce the total number of animals needed. The minimum numbers of animals in each group were determined using power calculations and data from previous experiments.

### Chronic Hypoxia-Induced Mouse Model of PH

The experimental protocol is depicted in FIG. 12A. Male C57BL/6J mice (Charles River) at the age of 10 weeks were either placed into a hypoxic chamber (FiO2=0.1, n=16) for 4 weeks, or kept at normoxic conditions (control group, FiO₂=0.21, n=8). According to the reports of Fagan et al. (Fagan, K A, et al. Am. J. Physiol. Lung Cell. Mol. Physiol. (2004) 287, L656-664) and Tuscherer et al. (Tuchscherer, H A, et al., J. Biomech. (2007) 40, 993-1001), a significant increase in the right ventricular systolic pressure (RVSP) and PA muscularization can be observed already after 14 days of hypoxia exposure. Therefore, mice subjected to hypoxia were randomized into two groups at the end of the second week. Eight mice received a subcutaneous slow-release pellet containing benzbromarone (Innovative Research of America, Sarasota Fla., USA) (HOX+BBR) and the other 8 mice received pellets with vehicle (HOX+Veh). The pellets ensured a stable blood concentration of the drug/vehicle over the next two weeks. At the end of week 4, all mice were subjected to in vivo hemodynamic measurements, followed by sacrifice and organ collection.

### Monocrotaline Treated Rats

The experimental protocol is shown in FIG. 13A. Male Sprague-Dawley rats (body weight: approx. 250 g, Charles River) received a single subcutaneous injection of monocrotaline (MCT, 60 mg/kg, n=16) or vehicle (control group, n=8). Since significant RVSP increase and PA muscularization had been detected as early as 12 days after the injection of MCT58, two weeks after monocrotaline treatment rats were randomly ordered into two groups. Subcutaneous slow-release pellets (Innovative Research of America) that contained either benzbromarone (MCT+BBR, n=8) or vehicle (MCT+Veh, n=8) were implanted. Four weeks after monocrotaline treatment all rats were subjected to echocardiography and in vivo hemodynamic measurements, thereafter they were sacrificed for organ collection.

### Echocardiography

Echocardiographic measurements were performed using a Vevo 770 High Resolution Imaging System with a 30 MHz RMV-707B scan head (VisualSonics, Toronto, Canada) as previously reported (Egemnazarov, B, et al., J. Am. Soc. Echocardiogr. (2015) 28, 828-843). Briefly, animals were mounted on a heated pad and kept under anaesthesia with isoflurane 0.8-1.2%. Chest hair was depilated and a layer of sonographic coupling gel was applied to the thorax. RV internal diameter (RVID) and free wall thickness (RVFW) from the left parasternal long axis view were measured in M-mode and 2-D modalities. RV outflow tract was visualized from the parasternal short axis view at the level of the aortic valve. From this view, pulse wave (PW) Doppler flow recordings of the pulmonary artery were obtained with the sample volume positioned at the tip of the pulmonary valve leaflets. Here, peak velocity, acceleration time (PAAT), and velocity-time integral (VTI) were measured. For every parameter, measurements were done for 4-5 cardiac cycles and results were averaged. Cardiac output was calculated as CO (mL/min)=(7.85×[RV outflow tract]2×pulmonary valve velocity-time integral×heart rate)/1,000. Cardiac index (CI) was obtained by normalizing cardiac output to body weight. The operator was blinded to the experimental group: animals were identified with number codes that did not carry information about the previous treatment.

### In Vivo Hemodynamics

### Mice

In mice, in vivo hemodynamics was performed under constant inhalation of 2% isoflurane/oxygen, using the closed chest technique through a small incision on the submandibular area. Body temperature was monitored and maintained at 37±1° C. A limb-lead ECG was recorded and the heart rate was kept constant to avoid changes in the sympathetic tone during the experiment. The right ventricle (RV) was catheterized via the right jugular vein using a 1.4 F Millar catheter (SPR-671, Millar, Houston Tex., USA) and right ventricular systolic pressure (RVSP) was measured continuously. Another catheter was inserted into the left carotid artery to monitor systemic blood pressure. After recording of stable values, the experiment was either continued with the assessment of the acute hemodynamic effects of benzbromarone (described above), or mice were sacrificed immediately for organ collection. Animals were identified with a number code throughout the experiment to ensure that the operator was blinded for the experimental groups.

### Acute Hemodynamic Effects of Benzbromarone in Mice

Mice of the above-described control and HOX+Veh groups were used for these studies. Benzbromarone was injected into the left jugular vein as a single bolus (300 μm, i.v. in 50 μl saline) and pressures were monitored until a maximum effect was observed (approx. 10 minutes post-injection). Only those mice that maintained a stable RVSP both before and 10 min after the BBR application were included in the analysis.

### Rats

In rats, in vivo hemodynamic measurements were conducted as described above for the mice, with the exception that for the right ventricular and the systolic blood pressures, 2 F Millar Catheters (SPR-513 and SPR-320, respectively) were used.

### Acute Hemodynamic Effects of Benzbromarone in Rats

The above described control and MCT+Veh groups were included in this study. Benzbromarone was injected into the left jugular vein as a single bolus (300 μm, i.v. in 100 μl saline) and pressures were monitored until a maximum effect was observed (approx. 10 minutes). Rats that failed to maintain a steady-state RVSP either before or after BBR injection were excluded from this analysis.

### Assessment of Right Ventricular Hypertrophy

At the end of the hemodynamic measurements, animals were exsanguinated and the heart and lungs were isolated. The right ventricle (RV) was dissected from the left ventricle+septum (LV+S) and both of them were weighed. Right ventricular hypertrophy was assessed by calculating the Fulton index, defined as RV weight/(LV+S weight).

### Immunohistological Assessment of Vascular Remodeling and Cell Proliferation

Paraffin-embedded mouse and rat lung tissues were cut to 2 μm thin sections. Double immunohistochemical staining and quantification of the non-muscularized and muscularized arteries were made as previously described (Crnkovic, S, et al., Respir. Physiol. Neurobiol. (2011) 179, 342-345). Immunostained slides were scanned with an Olympus BX61VS microscope using the OlyVIA software (Olympus Austria GmbH, Vienna, Austria). Vessel remodeling in lung sections was quantified using semi-automated image analysis software (Visiopharm, Hoersholm, Denmark). Throughout the analysis, all samples were identified with number codes to blind the operator for the experimental group. The percentage of non-muscularized and muscularized pulmonary arteries, relative to the total number of pulmonary arteries, was calculated.

Serial sections from the same lungs were used to determine cell proliferation in the PA wall, as described by Zabini et al. (Zabini, D. et al., J. Cell. Mol. Med. (2015) 19, 1151-1161). Lung sections were labeled using a primary antibody against the proliferation marker PCNA (sc-7907, Santa Cruz). Immunostained slides were scanned with an Olympus BX61VS microscope equipped with the OlyVIA software (Olympus Austria GmbH, Vienna, Austria). Negative controls were performed with the omission of the primary antibody.

### Assessment of the Hemodynamic Effect of a Single Oral Dose of Benzbromarone in IPAH Patients

The study protocol (Clinical trial registration number: NCT02790450; EudraCT number: 2015-000709-38) was approved by the Ethics Committee of the Medical University of Graz. According to the convention for human pharmacological pilot studies, 10 IPAH patients were involved; for ethical reasons, we did not establish a placebo group in this study. All patients provided a written informed consent to participate in the study. Examinations were performed by the same experienced team. For the right heart catheterizations, a 7F quadriple-lumen, balloon-tipped, flow-directed Swan-Ganz catheter (Baxter, Deerfield, Ill.) was introduced using the transjugular approach. Hemodynamic measurements included systolic, diastolic and mean pulmonary arterial pressure, pulmonary arterial wedge pressure, right atrial pressure, and cardiac output measured by the thermodilution technique and calculated using an analog computer system. Pulmonary vascular resistance was derived from the difference of mean pulmonary arterial pressure and pulmonary arterial wedge pressure divided by cardiac output. Cardiac index was determined as the ratio of cardiac output to body surface area. All measurements were performed in the supine position, pressure values were continuously recorded and averaged over several respiratory cycles during spontaneous breathing. Zero reference level was set at mid-thoracic level where the 4th rib inserts to the sternum (Kovacs, Avian et al. 2014). Partial pressure of oxygen and oxygen saturation of arterialized ear lobe capillary blood and pulmonary arterial blood were determined with an ABL 800 Flex (Radiometer; Copenhagen, Denmark) blood gas analyzer. Systemic blood pressure was measured by a sphygmomanometer.

### Assessment of CLCA1 Concentration in Human Plasma and Lung Homogenate

Plasma samples from 10 IPAH patients at the time of right heart catheterization, as well as from 10 age- and sex matched healthy controls were collected. A written informed consent was obtained from each patient prior to the interventions. The study was carried out under the clinical trial registration number NCT01607502, and was approved by the Ethics Committee of the Medical University of Graz. Samples were diluted 1:5 with PBS and the concentration of CLCA1 was determined using the Human CLCA1 ELISA Kit (Abbexa, Cambridge, UK). In order to determine the CLCA1 concentration in lung homogenates, frozen lung tissue pieces from 8 IPAH patients and 8 healthy donors were homogenized on liquid nitrogen and dissolved in PBS. A freeze-thaw cycle and sonication were applied to disrupt the cell membranes, followed by a centrifugation at 8000 g for 5 min. The supernatants, without dilution, were analyzed as described above.

### Statistical Analyses

Data are shown either as individual data plots with median, or summarized as mean±s.e.m. In the case of summarized data, n numbers for each group are given in the corresponding figure legend. Statistical analyses were performed using Prism 6.0 (GraphPad Software, La Jolla, Calif., U.S.), statistical tests were selected to be appropriate for the given dataset, and are identified in the corresponding figure legend. All datasets met the assumptions of the statistical test used and compared groups had similar variance. For all datasets, statistical analyses were two-sided, and P values <0.05 were considered significant.

1. A method of assessing the occurrence or the risk of occurrence of pulmonary hypertension (PH) in a subject, the method comprising detecting the expression level of the channel TMEM16A in a sample from the subject, wherein an increased level of TMEM16A expression, relative to a threshold value, indicates a risk of occurrence of PH or a likelihood of PH in the subject.

2. The method of claim 1, wherein the sample is a pulmonary arterial smooth muscle sample.

3. The method of claim 1, wherein the threshold value is based on a reference sample.

4. A method of identifying a compound capable of modulating activity of TMEM16A, the method comprising contacting in vitro or ex vivo TMEM16A with a compound suspected to modulate activity of TMEM16A, and detecting the activity of the TMEM16A.

5. The method of claim 4, wherein the TMEM16A is comprised in a host cell, wherein the host cell is optionally a pulmonary arterial smooth muscle cell.

6. The method of claim 4, wherein detecting the activity of TMEM16A comprises comparing the activity of TMEM16A to a control measurement and/or to a threshold value.

7. The use of a nonhuman animal to screen an agent for activity in treating PH, wherein the nonhuman animal has been exposed to hypoxic conditions for at least 10 days.

8. An agent suspected or known to reduce activity of TMEM16A for use in a screening method for a compound for treating PH, the use comprising administration of the agent to a non-human animal that has been exposed to hypoxic conditions for at least 10 days, and determining the right ventricular systolic pressure (RVSP), wherein a decrease in RVSP indicates that the compound is effective in treating PH.

9. An agent effective in reducing activity of TMEM16A for use in a method of treating PH in a subject.

10. The agent of claim 9, wherein the agent is identifiable by a method comprising contacting in vitro or ex vivo TMEM16A with a compound suspected to modulate activity of TMEM16A, and detecting the activity of the TMEM16A.

11. The agent of claim 8, being comprised in a pharmaceutical composition.

12. The method of claim 1, wherein the PH is pulmonary arterial hypertension (PAH).

13. The method of claim 12, wherein the PAH is Group 1 PAH.

14. The method of claim 13, wherein the Group 1 PAH is:
idiopathic or primary pulmonary hypertension,
familial hypertension,
pulmonary hypertension secondary to connective tissue disease, congenital heart defects (shunts), pulmonary fibrosis, portal hypertension, HIV infection, sickle cell disease, a drug and/or a toxin (e.g., anorexigens, cocaine), chronic hypoxia, chronic pulmonary obstructive disease, sleep apnea, and schistosomiasis,
pulmonary hypertension associated with significant venous or capillary involvement (pulmonary veno-occlusive disease, pulmonary capillary hemangiomatosis),
secondary pulmonary hypertension that is out of proportion to the degree of left ventricular dysfunction, or
persistent pulmonary hypertension in a newborn baby.

15. The method of claim 1, wherein the subject is human.


SEQUENCE LISTING (46 sequences)

SEQ ID NO: 1 - DNA, 20 bp, Artificial Sequence
  Primer
cacgatgagggtcaacgaga

SEQ ID NO: 2 - DNA, 20 bp, Artificial Sequence
  Primer
ataaggagttcagcagcgtg

SEQ ID NO: 3 - DNA, 24 bp, Artificial Sequence
  Primer
gggttgaaccatgattccggtgac

SEQ ID NO: 4 - DNA, 24 bp, Artificial Sequence
  Primer
gaagacggcaatcatcagcatgac

SEQ ID NO: 5 - DNA, 24 bp, Artificial Sequence
  Primer
acctggatggcccacaggtaatag

SEQ ID NO: 6 - DNA, 24 bp, Artificial Sequence
  Primer
atgctggtcaactggaacctctgc

SEQ ID NO: 7 - DNA, 19 bp, Artificial Sequence
  Primer
acaagccatgagcagcgac

SEQ ID NO: 8 - DNA, 20 bp, Artificial Sequence
  Primer
ggaatcatgtttctccgggc

SEQ ID NO: 9 - DNA, 24 bp, Artificial Sequence
  Primer
atggaggagtgaagtctcctgtcg

SEQ ID NO: 10 - DNA, 24 bp, Artificial Sequence
  Primer
cttccgcaagggtaaacattcctg

SEQ ID NO: 11 - DNA, 22 bp, Artificial Sequence
  Primer
accgtggccatgctcatgattg

SEQ ID NO: 12 - DNA, 23 bp, Artificial Sequence
  Primer
atcttgtcctgtgtcacctggag

SEQ ID NO: 13 - DNA, 21 bp, Artificial Sequence
  Primer
tgaacctcttcgtggctacat

SEQ ID NO: 14 - DNA, 23 bp, Artificial Sequence
  Primer
gcaagtgatgcatggaatactga

SEQ ID NO: 15 - DNA, 20 bp, Artificial Sequence
  Primer
gctcagttgggtggactacg

SEQ ID NO: 16 - DNA, 21 bp, Artificial Sequence
  Primer
cagctcttcctcaaacagtgc

SEQ ID NO: 17 - DNA, 20 bp, Artificial Sequence
  Primer
aagccggagtgagcctatga

SEQ ID NO: 18 - DNA, 20 bp, Artificial Sequence
  Primer
gctgtgttcccctgtgatgg

SEQ ID NO: 19 - DNA, 20 bp, Artificial Sequence
  Primer
atgaagccggaccagtccaa

SEQ ID NO: 20 - DNA, 20 bp, Artificial Sequence
  Primer
ggaggacctggcatgttgag

SEQ ID NO: 21 - DNA, 20 bp, Artificial Sequence
  Primer
acccaggagatcttcgcctt

SEQ ID NO: 22 - DNA, 20 bp, Artificial Sequence
  Primer
gtccacctctgagctgttgg

SEQ ID NO: 23 - DNA, 20 bp, Artificial Sequence
  Primer
ttaatgcccttcggcgatgt

SEQ ID NO: 24 - DNA, 21 bp, Artificial Sequence
  Primer
agcgttcctccttgttatccg

SEQ ID NO: 25 - DNA, 19 bp, Artificial Sequence
  Primer
ggatgcttcttccgaccct

SEQ ID NO: 26 - DNA, 21 bp, Artificial Sequence
  Primer
gaactgttaatgctgcctcca

SEQ ID NO: 27 - DNA, 21 bp, Artificial Sequence
  Primer
caggtaccaatcctccgttcc

SEQ ID NO: 28 - DNA, 20 bp, Artificial Sequence
  Primer
tcaggtgagggtaccttgga

SEQ ID NO: 29 - DNA, 18 bp, Artificial Sequence
  Primer
tgcacatcgtcgacacgg

SEQ ID NO: 30 - DNA, 20 bp, Artificial Sequence
  Primer
tgtgcggacacgtgtatttg

SEQ ID NO: 31 - DNA, 20 bp, Artificial Sequence
  Primer
ctgaatgggctgaaggagga

SEQ ID NO: 32 - DNA, 22 bp, Artificial Sequence
  Primer
atgaagagcctctgggaaaagg

SEQ ID NO: 33 - DNA, 20 bp, Artificial Sequence
  Primer
aacgacgtgtacaaaggcca

SEQ ID NO: 34 - DNA, 20 bp, Artificial Sequence
  Primer
atcgtgcagtgggtatgcag

SEQ ID NO: 35 - DNA, 20 bp, Artificial Sequence
  Primer
gtacagcatgggcatcacga

SEQ ID NO: 36 - DNA, 20 bp, Artificial Sequence
  Primer
aggtcgatgggctggtactt

SEQ ID NO: 37 - DNA, 20 bp, Artificial Sequence
  Primer
tctgtcttcatggccctctg

SEQ ID NO: 38 - DNA, 21 bp, Artificial Sequence
  Primer
atgatccttgacagcctcctc

SEQ ID NO: 39 - DNA, 20 bp, Artificial Sequence
  Primer
cagccacctcttcgacaacc

SEQ ID NO: 40 - DNA, 25 bp, Artificial Sequence
  Primer
ggatgatcctcttcctcttcaaagc

SEQ ID NO: 41 - DNA, 21 bp, Artificial Sequence
  Primer
agagctgaatacgaagccaga

SEQ ID NO: 42 - DNA, 20 bp, Artificial Sequence
  Primer
gccatggctgtcttaaccct

SEQ ID NO: 43 - DNA, 22 bp, Artificial Sequence
  Primer
tcctagagctgaatacgaagcc

SEQ ID NO: 44 - DNA, 24 bp, Artificial Sequence
  Primer
tgtcagtctctttgtttctggact

SEQ ID NO: 45 - DNA, 20 bp, Artificial Sequence
  Primer
tggaggctatccagcgtact

SEQ ID NO: 46 - DNA, 20 bp, Artificial Sequence
  Primer
tgtcggatggatgaaaccca